(19) United States
(12) Patent Application Publication
(10) Pub. No.: US 2024/0075194 A1
(43) Pub. Date: Mar. 7, 2024

(54) PERIPHERAL VASCULAR ACCESS FOR BLOOD FILTRATION SYSTEMS

(71) Applicant: Nuwellis, Inc., Eden Prairie, MN (US)

(72) Inventors: David Lerner, St. Paul, MN (US); David J. Haskvitz, Maple Grove, MN (US)

(73) Assignee: Nuwellis, Inc., Eden Prairie, MN (US)

(21) Appl. No.: 18/261,586

(22) PCT Filed: Mar. 1, 2022
(86) PCT No.: PCT/US2022/070887

§ 371 Date: Jul. 14, 2023

Related U.S. Application Data
(60) Provisional application No. 63/200,321, filed on Mar. 1, 2021.

(51) Int. Cl. A61M 1/34; A61B 17/135

(52) U.S. Cl. CPC A61M 1/3406; A61B 17/1355; A61M 2202/0021; A61M 2202/0028; A61M 2202/0415; A61M 2205/3303; A61M 2205/3306; A61M 2205/332; A61M 2205/3344; A61M 2205/3375; A61M 2205/50; A61M 2205/75; A61M 2209/00; A61M 2230/005; A61M 2230/20; A61M 2230/30

(57) ABSTRACT

A blood filtration system may include a blood circuit. The blood filtration system may include an adjustable cuff configured to receive a portion of a limb of a patient. The cuff may selectively engage with the limb to apply an external force to the limb. Applying force to the limb with the cuff may inhibit flow of blood within the vasculature of the limb. The system may adjust the cuff to change the force applied to the limb, for example to correspondingly change flow of blood from the vasculature to the blood circuit. The blood filtration system may include a controller. The controller may communicate with the cuff. The controller may operate the cuff to adjust the force applied to the limb of the patient.

### CLAIM OF PRIORITY

This patent application claims the benefit of priority of Lerner et al., U.S. Provisional Patent Application Ser. No. 62/200,321, entitled “PERIPHERAL VASCULAR ACCESS FOR BLOOD FILTRATION SYSTEMS,” filed on Mar. 1, 2021 (Attorney Docket No. 4567.040PRV), which is hereby incorporated by reference herein in its entirety.

### TECHNICAL FIELD

This document pertains generally, but not by way of limitation, to a blood filtration system.

### BACKGROUND

A blood filtration system may remove blood from the blood stream (e.g., venous circulation) of a patient and separate plasma water and electrolytes from erythrocytes (e.g., red blood cells) and other blood constituents with a filter. The system may convey the plasma water to a reservoir (e.g., a bag) for disposal. The balance of the plasma water, the erythrocytes, and other blood constituents are returned to the patient's blood stream. Once blood is withdrawn from the blood stream of a patient and the blood contacts extracorporeal components of the blood filtration system (e.g., tubing, the filter, or the like), a potential exists for clots to form within the extracorporeal components, leading to an increase in resistance within the components (e.g., the filter), and potentially clogging (e.g., occluding) the components. While not harmful to the patient, the increase in resistance or clotting could necessitate replacement of one or more of the extracorporeal components.

### SUMMARY

The present inventors have recognized, among other things, that a problem to be solved may include enhancing blood flow through a blood filtration system. In another example, the present inventors have recognized, among other things, that a problem to be solved may include minimizing recirculation of blood within a blood filtration system. The blood filtration system may include a blood circuit. The blood circuit is configured to transmit blood through the blood filtration system. For instance, a withdrawal line may receive blood of a patient. The withdrawal line may communicate with a filter. The filter may remove one or more plasma constituents from the blood of the patient. In another example, the blood circuit may include an infusion line. The infusion line may transmit blood (e.g., filtered blood, or the like) to the patient (e.g., by infusing blood into vasculature of the patient).

In some approaches, flow of blood within vasculature of the patient may be inhibited, such as when a patient flexes its arm. The patient flexing its arm may reduce blood flow available for withdrawal into the blood circuit. A reduction in blood flow within vasculature of the patient (or within the blood circuit) may increase resistance (to flow of blood) within the blood circuit.

A reduction in the flow of blood within the vasculature of the patient may cause stasis of the blood which may lead to clotting of the blood within the blood circuit. Clotting of blood may clog the blood circuit (or cause loss of the blood circuit).

In another approach, if the infusion and withdrawal lines 104, 106 are too close together in the vasculature of the patient, the filtered blood infused by the line 160 may be immediately withdrawn from the vasculature and into the line 104. Accordingly, filtered blood may be recirculated within the blood filtration system (e.g., blood is drawn into the withdrawal line after infusion rather than moving proximally in the vein and back toward the heart where it may mix with blood from other anatomic sites). For instance, the filter may remove plasma water from the blood. Recirculation may remove plasma water, and accordingly partially dehydrate the blood. Circulation of partially dehydrated blood within the blood circuit may increase the resistance to flow of blood within the blood circuit. Accordingly, circulation of partially dehydrated blood within the blood circuit may clog components of the blood circuit (e.g., the filter, or the like).

The present subject matter may help provide a solution to these problems, such as by providing a blood filtration system. For instance, the blood filtration system may include one or more of a withdrawal line or an infusion line configured to transmit blood from vasculature (e.g., a brachial vein, basilic vein, axillary vein, subclavian vein, or the like) of a patient. One or more of the withdrawal line or the infusion line may couple with a filter. In an example, the filter may remove one or more plasma constituents from the blood of the patient. For instance, the filter may remove plasma water from the blood of the patient. In another example, the blood filtration system may include a cuff. The cuff may inhibit flow of blood within a limb of the patient. For instance, the cuff may include one or more of a tourniquet, strap, sleeve, band, belt, constrictor, sphygmomanometer, or the like).

The cuff may be attached to a patient, for instance attached to a limb of the patient. The cuff may be tightened or loosened to change blood flow within vasculature proximate the cuff. In some examples, the cuff is attached to the limb of the patient and then tightened to apply a force to the limb. The cuff may apply a force (or a pressure corresponding to the force) to the limb of the patient. For instance, the cuff may include a bladder. Inflation of the bladder may correspondingly apply a force (or pressure) to the limb. In an example, the system may adjust the force (or pressure) applied to the limb, for instance by inflating (or deflating) the bladder. In another example, a strap may be mechanically or electromechanically controlled to vary tightness of the strap. Accordingly, the cuff may engage with (e.g., squeeze, cinch, constrict, compress, choke, grip, press, or the like) the limb to inhibit blood flow within the limb.

As described herein, the cuff may be attached to the limb of the patient. In an example, the withdrawal line may be located distally of the cuff. The infusion line may be located proximally of the cuff. For instance, the cuff may be attached to an arm proximate an elbow. The withdrawal line may be in communication with a first catheter, and the first catheter may be inserted into vasculature proximate a wrist of the arm (e.g., proximate a radius, or the like). The infusion line may be in communication with a second catheter, and the second catheter may be inserted into vasculature proximate to a bicep of a patient (e.g., proximate a humerus, or the like).

The cuff may be adjusted (e.g., tightened, or the like), for instance to inhibit flow within the limb. In another example, the cuff may include an inflated configuration and a deflated configuration. For instance, the cuff may include a shell. The shell may be rigid (however the present subject matter is not so limited). The cuff may include a bladder, and the bladder may be inflated (or deflated), for instance to engage the bladder with a limb. The cuff may include a limb socket, and the bladder may engage with a limb received in the limb socket. For example, the bladder may apply a force (or apply pressure) to the limb.

The system may inflate the bladder to vary the force applied to the limb. For instance, the system may include a pump. The pump may communicate with the bladder, for instance to provide a fluid (e.g., gas, liquid, or the like) to the bladder. In an example, communicating fluid with the bladder may include (but is not limited to) pumping, pushing, forcing, or sucking fluid into (or out of) the bladder. The bladder may be deflated to vary the force applied to the limb. For example, the fluid (or a portion of the fluid) may be removed from the bladder. In an example, the fluid is released from the bladder to decrease the force applied to the limb. In another example, fluid is released from the bladder in accordance with comfort of the patient (e.g., the cuff may be loosened to increase the comfort of the patient).

The cuff may inhibit blood flow within the limb, for instance by applying force (or pressure) to the limb. In an example, the cuff may inhibit venous flow (e.g., venous return, proximal blood flow, or the like) within the limb. The cuff may permit arterial flow (e.g., arterial inflow, distal blood flow, or the like) within the limb. Accordingly, the cuff may facilitate pooling of blood within the limb, for instance pooling of blood distal to the cuff. Thus, pressure in the limb (e.g., venous pressure, arterial pressure, or the like) may increase distal to the cuff. The increase in pressure within the limb may drive blood into the blood circuit. For example, the withdrawal line may communicate with vasculature distal to the cuff. The increase in pressure distal to the cuff may drive blood into the withdrawal line (and other portions of the blood circuit). Thus, the cuff may enhance blood flow in the limb for transmission through the blood circuit.

In yet another example, the blood filtration system may include a blood pump, and the blood pump may facilitate withdrawal of blood into the withdrawal line (e.g., by generating a negative pressure within the withdrawal line, or the like). The cuff may cooperate with the blood pump to transmit blood within the blood circuit. For example, the cuff may drive blood distally within the limb, and the blood pump may cooperate with the cuff to draw blood into the limb. The driving of blood into distal portion of the limb (and corresponding increase in pressure) may cooperate with the pressure generated by the blood pump to transmit blood through the blood circuit.

In another example, the cuff may minimize recirculation within the blood circuit. For instance, the cuff may inhibit flow within veins of the patient. In an example, the cuff is attached to an arm of the patient. The infusion line may communicate with vasculature located proximally with respect to the cuff. The withdrawal line may communicate with vasculature located distally to the cuff. Accordingly, in some examples, the withdrawal line may communicate with a first piece of vasculature (e.g., an ulnar vein, radial vein, basilic vein, or the like). The infusion line may communicate with a second piece of vasculature (e.g., a brachial vein, axillary vein, cephalic vein, or the like). Thus, recirculation of (filtered) blood from the infusion line to the withdrawal line may be reduced by withdrawing blood from a different portion of vasculature than the infusion lumen. In another example, the infusion lumen may be proximal with respect to the withdrawal lumen. The (filtered) blood infused into the patient flows proximally toward the heart (and away from the withdrawal lumen). Thus, recirculation from the infusion line to the withdrawal line is reduced.

In still yet another example, the cuff may inhibit flow within the veins (e.g., flow of blood within the vein from a distal portion of a limb to a proximal portion of a limb). Thus, blood infused from the infusion line (located proximal to the cuff) is permitted to flow proximally, for instance toward a heart of the patient where the infused blood mixes with blood from other anatomical locations. The withdrawal line may be distal of the cuff, and the cuff may inhibit distal flow of infused blood to the withdrawal line. Accordingly, the blood filtration system minimizes recirculation of blood from the infusion line to the withdrawal line.

This overview is intended to provide an overview of subject matter of the present patent application. The overview is not intended to provide an exclusive or exhaustive explanation of the subject matter. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components.

The drawings illustrate generally, by way of example, but not by way of limitation, various embodiments discussed in the present document.

FIG. 1 shows a schematic view of an example of portions of a blood filtration system.

FIG. 2 shows an example of a cuff configured to attach to a limb of a patient.

FIG. 3 shows another example of the blood filtration system.

FIG. 4 shows the cuff in a loosened configuration.

FIG. 5 shows the cuff in a tightened configuration.

FIG. 6 shows a side view of the cuff optionally including a bladder.

FIG. 7 shows an end view of the cuff.

FIG. 8 shows an example of a method for operating a cuff configured to inhibit blood flow in a limb of a patient.

FIG. 9 shows a graphical representation of venous pressure in a limb with respect to time.

FIG. 10 shows an example of the cuff attached to a leg of a patient.

FIG. 11 shows a block diagram of an example machine upon which any one or more of the techniques (e.g., methodologies) discussed herein may perform.

FIG. 12 shows one example of a method for operating a blood filtration system.

### DETAILED DESCRIPTION

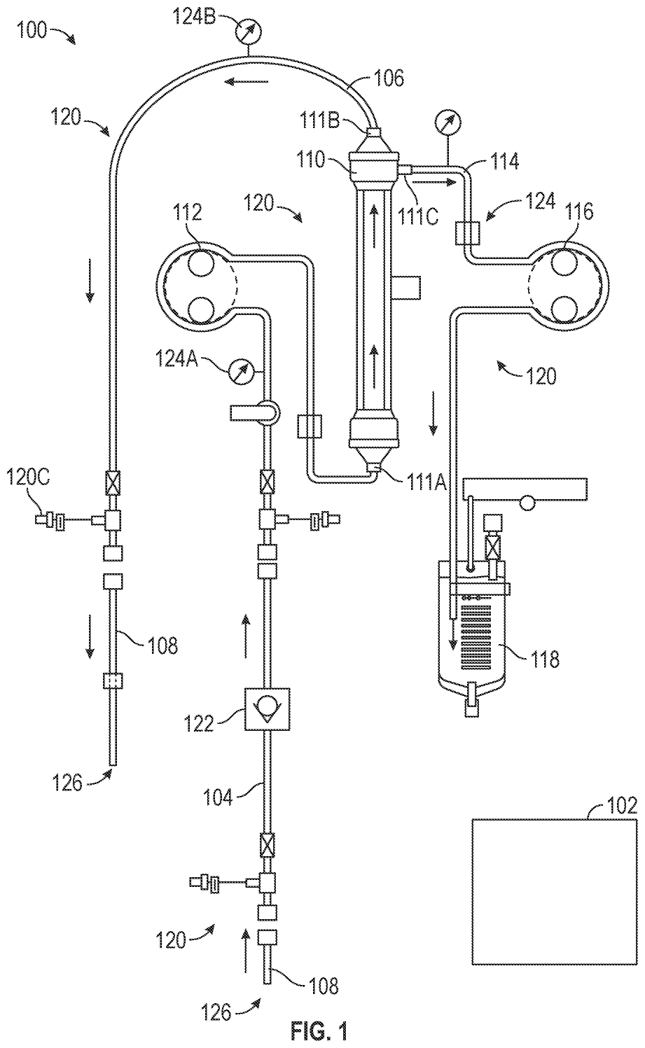

FIG. 1 shows a schematic view of an example of portions of a blood filtration system 100, according to an embodiment of the present subject matter. The blood filtration system 100 may reduce one or more plasma constituents (e.g., water, proteins, electrolytes, or the like) in blood of a patient. The blood filtration system 100 may facilitate one or more blood filtration operations, including (but not limited to): extracorporeal ultrafiltration, continuing renal replacement therapy (“CRRT”), slow continuous ultrafiltration (“SCUF”), continuous veno-venous hemofiltration (“CVVH”), continuous veno-venous hemofiltration (“CVVHD”), dialysis, continuous veno-venous hemofiltration including dialysis and filtration (“CVVHDF”), sustained low efficiency dialysis (“SLED”), extracorporeal membrane oxygenation (“ECMO”) therapy, modified ultrafiltration, and peripheral plasmapheresis, peripheral hemofiltration.

The blood filtration system 100 may include a controller 102. The controller 102 may include processing circuitry, for instance an integrated circuit. As described herein, the controller 102 may be configured to control one or more components of the blood filtrations system 100.

The blood filtration system 100 may include a withdrawal line 104 and may include an infusion line 106. The lines 104, 106 may be configured to couple with a catheter 108, and the lines 104, 106 may transmit blood within the blood filtration system 100. In an example, the catheter 108 may be inserted into a blood stream of the patient, for instance the catheter 108 may be inserted into a basilic vein, cephalic vein, brachial vein, the axillary vein, the subclavian vein, the brachiocephalic vein, saphenous vein, femoral vein, or the like. Blood may flow into the catheter 108, into the withdrawal line 104, through other components of the system 100, through the infusion line 106, into the catheter 108, and back into the blood stream of the patient. The line 104 may be separate from the line 106. The lines 104, 106 may be in communication with the catheter 108. For example, the catheter 108 may include one or more lumens, for example a withdrawal lumen in communication with the line 104 and an infusion lumen in communication with the line 106.

The lines 104, 106 may be configured to couple with a filter 110, for instance the lines 104, 106 may include one or more fittings that facilitate coupling the lines 104, 106 with the filter 110. In an example, the withdrawal line 104 may couple with a filter inlet port 111A, and the infusion line 106 may couple with a filter outlet port 111B. The filter 110 may be configured to reduce an amount of one or more plasma constituents (e.g., water, electrolytes, or the like) in blood flowing through the filter 110 and provide a filtrate fluid including the one or more plasma constituents. As described herein, blood may flow through the lines 104, 106 to and from the catheter 108. The lines 104, 106 may be coupled with the filter and blood may flow from the withdrawal line 104, through the filter 110, and into the infusion line 106.

The blood filtration system 100 may include a blood pump 112, and the blood pump 112 may pump (e.g., convey, drive, push, or the like) blood through the blood filtration system 100. In an example, the blood pump 112 may be a peristaltic pump, and the blood pump 112 may engage with the withdrawal line 104 to pump blood through the withdrawal line 104 and into the filter 110. The controller 102 may be configured to operate the blood pump 112 to vary a speed of the blood pump 112 and accordingly vary the flow rate of blood through the blood filtration system 100 (e.g., the withdrawal line 104, the filter 110, the infusion line 106, or the like).

Referring to FIG. 1, the blood filtration system 100 may include a filtration line 114 and a filtration pump 116. The filtration line 114 may be configured to couple with the filter 110 (e.g., with a fitting), for instance the filtration line 114 may couple with a filtrate fluid port 111C. The filter 110 may be configured to transmit the filtrate fluid (including one or more plasma constituents) extracted by the filter 110 to the filtrate fluid port 111C.

The filtration pump 116 may pump extracted filtrate fluid from the filter 110, and into a filtrate fluid reservoir 118 (e.g., a bag, container, bladder, or the like). In some examples, the filtration pump 116 may be a peristaltic pump that engages with the filtration line 114 to pump the filtrate fluid through the filtration line 114. The controller 102 may be configured to vary a speed of the filtration pump 116 and accordingly vary the flow rate of filtrate fluid through the blood filtrate system 100 (e.g., the filtration line 114).

The system 100 may include a blood circuit 120, and the blood circuit 120 may include one or more components of the system 100, such as may provide a conduit for blood flow. For example, the blood circuit 120 may include (but is not limited to) the withdrawal line 104, the infusion line 106, the catheter 108, the filter 110, the filtration line 114, the filtrate fluid reservoir 118. The blood circuit 120 may include components of the system 100 that are in communication with a biological fluid of the patient.

In some approaches, when the withdrawn venous blood contacts the blood circuit 120, a potential exists for a clot to occur in the blood circuit 120. For example, blood may clot when flowing through the filter 110, and the clotting may occlude the filter 110. An occlusion in the blood circuit 120 may lead to an increase in resistance of flow of blood through the filter 110. Clotting of blood in the filter 110 may result in loss of the filter 110 (e.g., because the filter becomes clogged due to clotting of blood in the filter 110). Accordingly, clotting of the filter 110 may necessitate replacement with a new filter 110.

An anti-coagulant may be used to inhibit occlusion in the blood circuit, for instance to inhibit clots from forming in the filter 110. For example, an anti-coagulant is infused (e.g., with non-regional infusion) into the blood circuit 120. The anti-coagulant will be introduced into the circulatory system of the patient, for instance because the blood flowing through the blood circuit 120 is infused back into the patient. Introduction of anti-coagulant into the patient may result in unwanted outcomes (e.g., bleeding, heparin induced thrombocytopenia, or the like). Accordingly, the present subject matter reduces occlusion of the blood circuit 120 without requiring an anti-coagulant (or use in combination with an anti-coagulant). For instance, the cuff 200 may drive blood distally relative to the cuff 200 to reduce occlusion of the blood circuit 120.

FIG. 1 shows the withdrawal line 104 may be in communication with a first characteristic sensor 124A and the infusion line 106 may be in communication with a second characteristic sensor 124B. The sensors 124A may determine pressure in the withdrawal line 104, and the sensor 124B may determine pressure in the infusion line 106. The sensors 124A, 124B may be in communication with the controller 102, and the controller 102 may determine the pressure in the lines 104, 106 using the sensors 124A, 124B.

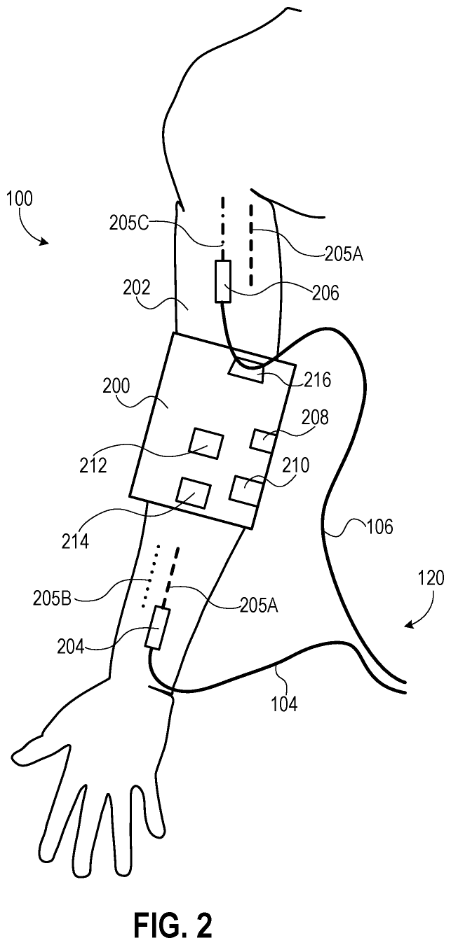

FIG. 2 shows an example of a cuff 200 configured to attach to a limb 202 (e.g., an arm, leg, or the like) of a patient. The cuff 200 may be located proximate a joint of the limb 202. In an example, the cuff 200 may be located proximate an elbow of the limb 202. In another example, the cuff 200 may be located proximate a cubital fossa of the limb 202. In an example, the cuff 200 may be attached to the patient superior to (or inferior to) the cubital fossa of the patient. The cuff 200 may be located proximate a knee of the limb 202. For instance, the cuff may be located proximate a popliteal fossa of the limb 202. The cuff 200 may be attached to a thigh of a patient. The cuff 200 may be located proximate a wrist of the limb 202. The cuff 200 may be located proximate an ankle of the limb 202.

The cuff 200 may inhibit flow of blood in the limb 202 of the patient. For instance, the cuff 200 may inhibit venous return within the limb 202. The cuff 200 may inhibit proximal blood flow in the limb 202. The cuff 200 may permit arterial inflow into the limb. The cuff 200 may permit distal blood flow in the limb 202. Accordingly, blood may pool within the limb distal to the cuff 200. Thus, pressure in the limb (e.g., venous pressure, arterial pressure, or the like) may increase distal to the cuff 200. The increase in pressure within the limb 202 may drive blood into the blood circuit 120. Thus, the cuff 200 may enhance blood flow in the limb 202 for transmission through the blood circuit 120.

In an example, the withdrawal line 104 may be in communication with a withdrawal catheter 204. The catheter 204 may supply blood to the withdrawal line 104. The infusion line 106 may be in communication with an infusion catheter 206. The catheter 206 may supply blood to the infusion line 106. The catheters 204, 206 may be inserted into vasculature of a patient, for instance to transmit blood within the vasculature through the blood circuit 120. In some examples, one or more of the withdrawal catheter 204 or the infusion catheter 206 include a needle catheter. In another example, the catheters 204, 206 may be single lumen catheters. In yet another example, the catheters 204, 206 may be included in a dual lumen catheter.

The catheters 204, 206 may be located in the same vein. In an example, the catheters 204, 206 may communicate with a vein, including (but not limited to) a basilic vein. The catheter 204 may be located in a first vein 205A. The first vein 205A is shown in FIG. 2 with a dashed line. The catheter 206 may be located in the first vein 205A. In an example, the withdrawal catheter 204 may be inserted into the first vein 205A distal with respect to the cuff 200. The catheter 206 may be inserted into the first vein 205A proximal with respect to the cuff 200.

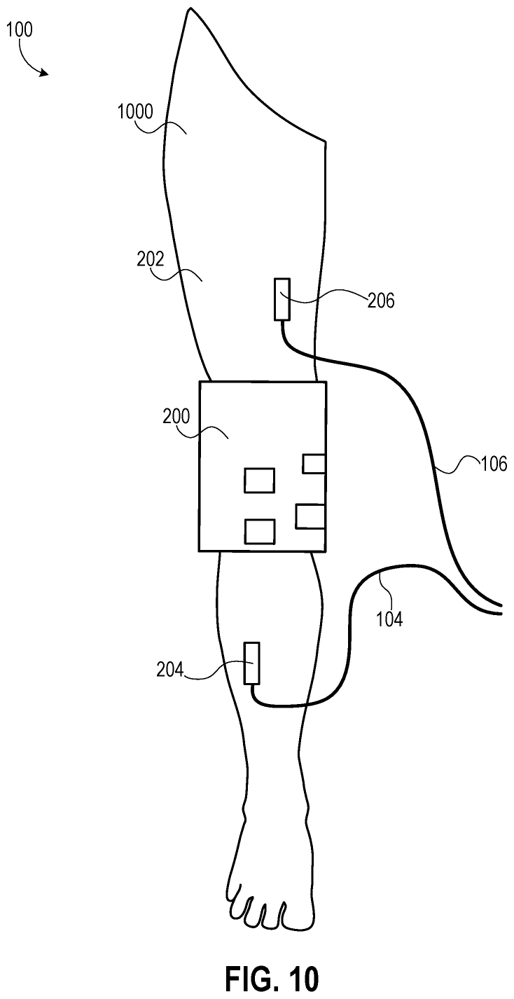

In another example, the catheters 204, 206 may communicate with different veins. The withdrawal catheter 204 may be located in a second vein 205B, including (but not limited to) a radial vein. The second vein 205B is shown in FIG. 2 with a dotted line. The infusion catheter 206 may be located in a third vein 205C (different than the second vein 205B), including (but not limited to) a cephalic vein. The third vein 205C is shown in FIG. 2 with a dot-dashed line. The first vein 205A may be included in a first limb (e.g., a left arm of a patient, a right leg of the patient, or the like), such as the arm shown in FIG. 2. The second vein 205B may be included in a second limb (e.g., a right arm of the patient, left leg of the patient, or the like), such as the leg shown in FIG. 10.

As described herein, the cuff 200 may inhibit flow of blood in the limb 202, for instance to drive blood proximally within the limb 202. The cuff 200 may inhibit proximal flow (e.g., venous return, or the like) of blood within the vasculature of the limb 202. In another example, the cuff 200 may inhibit flow of blood in vasculature by applying a force (e.g., an external force, ex vivo force, or the like) to the vasculature. The cuff 200 may inhibit flow by applying pressure (e.g., force per unit area, or the like) to the vasculature. The pressure applied by the cuff 200 (e.g., a cuff pressure, or the like) may exceed pressure in the vasculature, for example the venous pressure within a vein of the limb 202. Applying pressure (e.g., external pressure, ex vivo pressure, or the like) that exceeds the pressure within the vasculature may inhibit (e.g., occlude, stop, slow, reduce, minimize, or the like) blood flow within the vasculature. For instance, flow within vasculature is inhibited when the pressure applied by the cuff to the limb 202 is greater than (or equal to) the pressure within the vasculature.

Referring to FIG. 2, the cuff 200 may permit arterial inflow in the limb 202. The cuff 200 may permit distal flow of blood within the vasculature of the limb 202. For instance, the pressure applied by the cuff 200 may be less than pressure in arteries of the limb 202. The cuff 200 may permit distal flow of blood in the limb 202 (e.g., blood flow from the shoulder of a patient toward a fingertip of the patient, or the like). In another example with the cuff 200 inhibiting flow within veins (e.g., the first vein 205A, or the like) of the limb 202, the cuff 200 may inhibit flow of blood proximally within the limb 202. In an example, the cuff 200 may facilitate an increase in pressure within vasculature (e.g., the second vein 205B) distal of the cuff 200. The increase in pressure distal of the cuff 200 may drive blood into the withdrawal catheter 204, for instance because the cuff 200 permits arterial inflow while inhibiting venous return (e.g., flow within one or more veins of the limb 202, or the like). Accordingly, the cuff 200 may enhance blood flow within the blood circuit 120 by inhibiting blood flow within a portion of the limb 202. The system 100 may monitor physiological parameters to maintain sufficient arterial inflow to the limb.

In some examples, the cuff 200 includes a conduit 208 (e.g., a port, valve, passageway, fitting, coupling, or the like) that facilitates tightening or loosening of the cuff 200. Tightening (or loosening) of the cuff may vary the force (or pressure) applied to the limb 202 by the cuff 200. For instance, the cuff 200 may be inflated by pumping a fluid into the cuff 200 through the conduit 208. Accordingly, the cuff 200 may apply a pressure to the limb 202, for instance to inhibit venous return within the limb 202. The cuff 200 may be deflated, for example by pumping the fluid out of the cuff 200 through the conduit 208. The conduit may have an adjustable cross-section. For instance, the cross-section of the orifice may change in correspondence with the pressure in the bladder. Accordingly, the change in cross-section may facilitate a rate of deflation of the bladder (e.g., loosening of the cuff, or the like). Thus, the pressure applied by the cuff 200 may be reduced, for instance to allow venous return within the limb 202. In another example, the pressure applied by the cuff may be reduced to enhance comfort of the patient.

In another example, the cuff 200 includes a cuff characteristic sensor 210. The sensor 210 may measure one or more of a force, pressure, or the like generated by the cuff 200 to the limb 202. For example, the sensor 210 may measure pressure of fluid within the cuff 200. The sensor 210 may communicate with other components of the blood filtration system 100, such as the controller 102. The system 100 may operate the cuff 200 using the cuff characteristic sensor 210. For example, the system 100 may inflate the cuff 200 to a specified pressure (e.g., a pressure greater than a venous pressure of the patient, a pressure less than an arterial pressure of the patient, or the like).

Referring to FIG. 2, the cuff 200 may include a heating element 212. The heating element 212 may provide heat to the limb 202. For instance, the heating element 212 may enhance vasodilation of vasculature of the patient. Vasodilation of the vasculature enhances blood flow within the vasculature. Accordingly, the heating element may enhance blood flow within the vasculature (and enhance performance of the system 100) by applying heat to the limb 202. The system 100 may include a temperature sensor 214. For example, the cuff 200 may include the temperature sensor 214. The controller 102 may operate the heating element 212, for instance when the temperature measured by the sensor 214 exceeds (e.g., is less than, greater than, or the like) a temperature threshold. Thus, the controller 102 may enhance blood flow in the limb by heating the limb 202. In another example, the cuff 200 includes a guide 216 configured to receive one or more of the withdrawal line 104 or the infusion line 106.

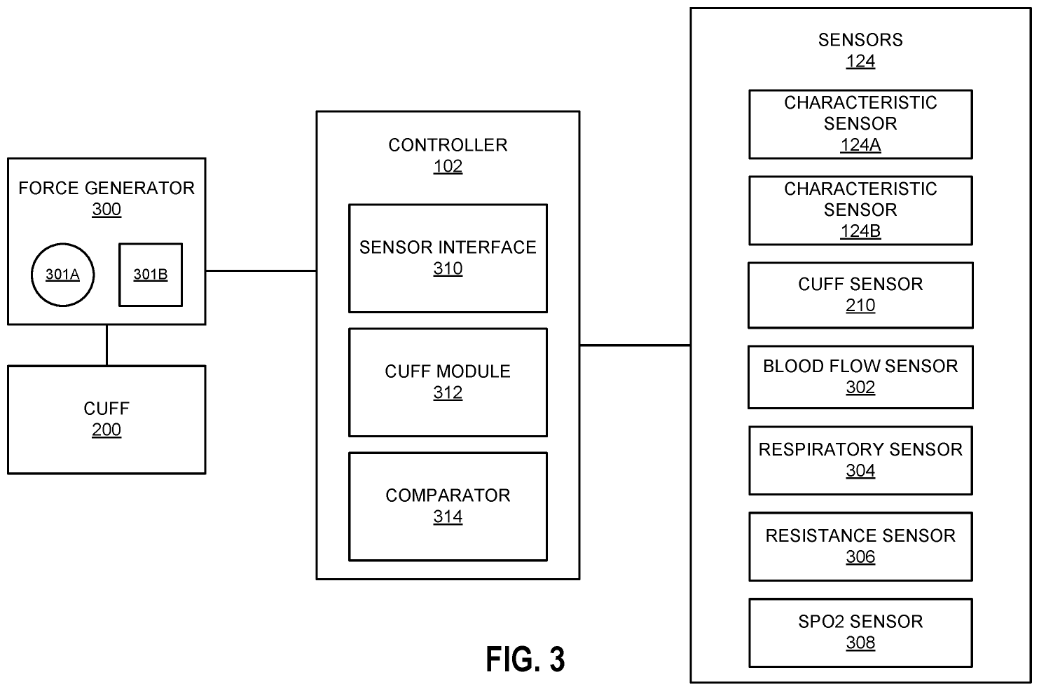

FIG. 3 shows another example of the blood filtration system 100, according to an embodiment of the present subject matter. The blood filtration system 100 may include the cuff 200. The system 100 may include a force generator 300. The force generator 300 may cooperate with the cuff 200 to apply a force to the limb 202. In an example, the conduit 208 (shown in FIG. 2) may be in communication with the force generator 300. The force generator 300 may cooperate with the cuff 200 to generate a force upon the limb 202 when the cuff 200 is attached to the limb 202. For instance, the force generated by the force generator 300 may act upon a surface of the skin of the patient. In an example, the force generator 300 tightens the cuff 200. For instance, the cuff 200 may tighten by inflating the cuff 200 and accordingly apply a force to the limb 202.

In an example, the force generator 300 includes a pump 301A (e.g., a diaphragm pump, a swing piston pump, bellows pump, syringe pump, or the like) that pumps a fluid into (or out of) the cuff 200. The pump 301A may pump one or more of a gas or a liquid into (or out of) the cuff 200 to generate the force applied to the limb 202. The cuff 200 may include mechanical, electromechanical, piezoelectric, or other components that facilitate generating the force applied to the limb 202 (shown in FIG. 2). In another example, a bladder is in communication with the force generator 300. The bladder may be displaced (e.g., expanded, contracted, or the like) to generate the force applied to the limb 202.

In another example, the force generator 300 includes an electrical switch 301B. The cuff 200 may tighten in accordance with operation (e.g., opening, closing, or the like) of the electrical switch 301B. For instance, the controller 102 may operate the switch 301B to tighten (or loosen) a band included in the cuff 200. The tightening of the band may inhibit blood flow in vasculature of a patient. Thus, the force generator 300 may include (but is not limited to) the electrical switch 301B, pump 301A, an actuator, a pneumatic cylinder, winch, spool, gear, servomotor, or the like, The controller 102 may operate the force generator 300 to adjust the force (or pressure) applied by the cuff 200. The controller 102 may operate the cuff 200 using the cuff sensor 210. For instance, the controller 102 may operate the cuff 200 to apply a pressure greater than (or equal to) the venous pressure of a vein within the limb 202. The controller 102 may operate the cuff 200 to apply a pressure less than the venous pressure of the vein. The controller 102 may operate the cuff 200 to apply a pressure less than an arterial pressure (e.g., systolic, diastolic, or the like) of an artery in the limb of the patient.

In an example, the controller 102 may operate (e.g., modulate, drive, activate, actuate, energize, or the like) the cuff 200 according to one or more physiological characteristics of a patient. For instance, the controller 102 may selectively tighten, or selectively loosen, the cuff 200 in accordance with one or more physiological characteristics of the patient. The controller 102 may adjust the force applied to the limb of the patient (e.g., by operating the force generator 300) in proportion to a change in the measured physiological characteristics.

In yet another example, the controller 102 may operate the cuff 200 according to one or more characteristics of the system 100. For instance, the system 100 (e.g., the controller 102 in communication with sensors 124) may determine venous pressure of the patient (e.g., pressure within a basilic vein, brachial vein, cephalic vein, or the like). A user may determine one or more of venous pressure or arterial pressure (e.g., systolic, diastolic, or the like). The controller 102 may receive user input of the determined venous pressure or arterial pressure. The system 100 may use the user-input venous pressure or arterial pressure to operate the cuff 200. The controller 102 may operate the force generator 300 (e.g., to pump fluid into the cuff 200) based on the measured venous pressure of the patient. For instance, the controller 102 may operate the force generator 300 to apply pressure (with the cuff 200) to the limb 202 that is greater than (or equal to) the determined venous pressure of the patient. For instance, the cuff 200 may apply pressure that is greater than (or equal to) the venous pressure of the patient. Accordingly, the cuff 200 may inhibit blood flow in the limb 202 (e.g., inhibiting blood flow such as venous return in the limb 202, or the like). In another example, the system 100 may operate the cuff 200 and the cuff 200 may apply pressure that is less than the arterial pressure of the patient. Thus, the cuff 200 may permit arterial inflow within the limb 202.

Referring to FIG. 3, and as described herein, the controller 102 may operate the cuff 200 based on blood flow of a patient (e.g., venous flow, arterial flow, or the like). For example, the system 100 may include a blood flow sensor 302 (e.g., a Doppler velocimeter, ultrasound transducer, or the like). The sensor 302 may determine (e.g., measure, quantify, check, gauge, assess, report, or the like) a blood flow rate in vasculature (e.g., the first vein 205A, shown in FIG. 2) of a patient. For instance, the blood flow sensor 302 may be located proximate the first vein 205A (shown in FIG. 2). The system 100 may use the blood flow sensor 302 to determine the rate of blood flow within the first vein 205A. For instance, the system 100 may determine venous flow in the limb 202 using the sensor 302. In an example, the blood flow sensor 302 may be located proximal along the limb 202 with respect to the withdrawal line 104 (however the present subject matter is not so limited).

The controller 102 may operate the cuff 200 based on the determined blood flow rate in the vasculature of the patient. For instance, the controller 102 may operate the force generator 300 to increase pressure applied by the cuff 200. The controller 102 may monitor blood flow in vasculature of the patient to determine when flow in the vasculature is inhibited by the cuff 200 (e.g., when blood flow rate is reduced, stopped, or the like). For instance, the controller 102 may maintain the pressure applied by the cuff 200 in correspondence with the controller 102 determining blood flow is inhibited by the cuff 200. Accordingly, the controller 102 may cooperate with the cuff 200 to increase pressure applied to the limb 202 to inhibit flow of blood within vasculature. For instance, the controller 102 may cooperate with the cuff 200 to apply a pressure that is lower than systolic pressure of the patient, and higher than the venous pressure.

The controller 102 may cease increasing pressure applied to the limb (e.g., by operating the force generator to stop increasing pressure, or the like) when blood flow in the vasculature is inhibited by the cuff 200 (e.g., when flow in a vein is reduced to zero flow, or the like). In an example, the pressure applied by the cuff 200 may equal the venous pressure of the patient. Accordingly, the cuff 200 may reduce vascular flow while minimizing pressure applied to the limb 202. For example, the system 100 may enhance patient comfort by limiting the force (or pressure) applied to the limb 202. The controller 102 may limit the pressure applied to the limb once the controller 102 determines flow in the vasculature is inhibited by the cuff 200. Accordingly, the system 100 may enhance patient comfort, for instance because the pressure applied by the cuff 200 is limited to the pressure corresponding to the pressure that inhibits blood flow in the limb 202.

The cuff 200 may be operated for a specified time period. For instance, the controller 102 may operate the cuff 200 to tighten the cuff 200 and inhibit blood flow in the limb 202 for a first time period (e.g., 10 seconds, 22 seconds, 60 seconds, or the like). The cuff 200 may be loosened (e.g., according to an electronic signal provided by the controller 102 to the force generator 300, or the like) to permit flow in the limb 202, for instance when the first period is exceeded. The controller 102 may operate the cuff 200 periodically (e.g., cyclically, or the like), for instance to selectively inhibit (or permit) blood flow in the limb 202.

As described herein, the controller 102 may operate the cuff 200 according to one or more physiological characteristics of a patient. For instance, the sensors 124 may determine one or more of the physiological characteristics of the patient. The system 100 may monitor the physiological characteristics using the sensors 124. For instance, the system 100 may monitor physiological parameters to maintain sufficient arterial inflow to the limb 202 (in correspondence with inhibiting venous return). In an example, the system 100 may determine a phase of a respiratory cycle (e.g., end inspiration, end expiration, or other phase) of a patient. The system 100 may include a respiratory sensor 304 (e.g., a sensor utilizing one or more of bioimpedance plethysmography, pneumatic plethysmography, photoelectric plethysmography, pulse oximetry, strain gage plethysmography, spirometry, or the like) to determine the phase of the respiratory cycle of the patient.

The blood filtration system 100 may operate the cuff 200 based on the phase of the respiratory cycle of the patient. In an example, the force applied to the limb 202 may be synchronized to the determined respiratory phase (e.g., using the controller 102 of the blood filtration system 100). For instance, the respiratory sensor 304 may determine when the patient inhales or exhales (e.g., a point in time when the patient stops inhaling). The controller 102 may communicate with the sensor 304. Accordingly, the system 100 may monitor the respiratory cycle of the patient and operate the cuff 200 based on the respiratory cycle.

The controller 102 may operate the cuff 200 based on resistance within one or more components of the blood circuit 120. For example, the system 100 may include a resistance sensor 306 (e.g., a pressure sensor, or the like) to determine the access resistance (e.g., a pressure sensor, or the like). The controller 102 may determine the access resistance using one or more of the sensors 124, such as a pressure sensor in communication with one or more of the lines 104, 106 (e.g., one or more of the sensors 124A, 124B). The controller 102 may compare the determined access resistance with a resistance threshold. The controller 102 may operate the force generator 300 based on the comparison of the determined access resistance to the resistance threshold (e.g., in correspondence with the determined access resistance exceeding the resistance threshold). The system 100 may include or more components, features, functions, or the like of the subject matter discussed in PCT application PCT/US2019/069130, which is hereby incorporated by reference herein in its entirety.

An increase in the resistance characteristic of the lines 104, 106 (or other components of the blood circuit 120) may diminish blood flow through the lines 104, 106. The resistance characteristic of the lines 104, 106 may be referred to as access resistance. The diminished blood flow due to the increase in the resistance characteristic of the lines 104, 106 may reduce the performance of the system 100, for example by reducing the maximum blood flow rate through the blood circuit 120. In another example, the diminished blood flow may reduce the rate that the one or more blood constituents may be removed from the blood by the filter 110. For instance, the plasma constituents may be removed through one or more of convection or diffusion in the filter 110. The resistance characteristic of the lines 104, 106 may increase to the point where the blood pump 112 is unable to maintain flow within the lines 104, 106 (e.g., because the forces resisting flow in the lines 104, 106 is greater than the forces generated by the blood pump 112). Accordingly, an increase in the resistance characteristic of the lines 104, 106 may diminish the performance of the blood filtration system 100. The controller 102 may operate the cuff 102 to drive blood to the withdrawal line 104. Thus, the system 100 may enhance blood flow through the blood circuit 120 using the cuff 200 to drive blood into the withdrawal line 104.

In another example, FIG. 1 shows the withdrawal line 104 may communicate with a characteristic sensor 124A. The infusion line 106 may communicate with a characteristic sensor 124B. The sensor 124A may determine pressure in the withdrawal line 104. The sensor 124B may determine pressure in the infusion line 106. The sensors 124A, 124B may communicate with the controller 102. Accordingly, the blood filtration system 100 may determine the pressure in the lines 104, 106 using the sensors 124A, 124B.

As described herein, controller 102 may operate the blood pump 112. The blood pump 112 may generate a negative pressure in the withdrawal line 104, for instance to withdraw blood from the vasculature in communication with the catheter tip 130 (shown in FIG. 1). The blood pump 112 may generate a positive pressure in the infusion line 106. For instance, the pump 112 may generate a positive pressure to infuse blood into the vasculature in communication with the catheter tip 130. The system 100 may increase the magnitude of the pressure in lines 104, 106 to correspondingly increase the flow rate of blood within the lines 104, 106. One or more of the catheter tips 130 associated with lines 104, 106 may be oriented with flow in the vasculature. One or more of the catheter tips 130 associated with the lines 104, 106 may be oriented against flow in the vasculature. In some examples, the catheter tip is located proximate the cuff 200. For instance, the catheter tip 130 may be located between the cuff 200 and the limb 202 of the patient. In an example, one or more of the withdrawal line 104 or the infusion line 106 extend through the cuff to facilitate withdrawing (or infusing) blood with the catheter tip 130 between the cuff 200 and the limb 202.

The system 100 may determine a total resistance characteristic for one or more components of the blood circuit 120. For example, the system 100 may determine the total resistance characteristic for one or more of the withdrawal line 104, the infusion line 106, the filter 110, or the blood circuit 120. In an example, a withdrawal line resistance characteristic may correspond to the resistance (to flow of blood) through the withdrawal line 104. The withdrawal line resistance characteristic may correspond with the pressure in the withdrawal line 104 divided by the actual blood flow rate of system 100 (e.g., as determined by a flow sensor in communication with the blood circuit 120). The actual blood flow rate of the system 100 may vary from a set point provided by the controller 102 to operate the blood pump 112. For example, the resistance characteristic of the lines 104, 106 may reduce the actual blood flow rate through the blood circuit 120 because the resistance to the flow of blood in the lines 104, 106 decreases the efficiency of the blood pump 112. Accordingly, the decreased efficiency of the blood pump 112 correspondingly decreases the actual blood flow rate through the blood circuit 120.

The infusion line resistance characteristic may correspond to the resistance in the infusion line 106 to the flow of blood through the infusion line 106. The infusion line resistance characteristic may correspond to the pressure in the infusion line 106 divided by the difference between the actual blood flow rate and the filtration rate of the system (e.g., as determined by controller 102 in communication with the sensors 124). An increase in the magnitude of the resistance characteristic of the blood circuit 120 (including the lines 104, 106) may increase the force necessary to withdraw blood from (or infuse blood into) the patient using the blood pump 112. The increase in the magnitude of the resistance characteristic of the blood circuit 120 may result in (or be an indication of) clotting in the blood circuit 120.

The cuff 200 may be operated according to one or more of the characteristics of the blood circuit 120. For example, the cuff 200 may be operated to inhibit flow within the limb 202 when the withdrawal resistance (e.g., withdrawal pressure divided by withdrawal blood flow rate) exceeds a resistance threshold. For instance, the system 100 may operate the cuff 200 to inhibit flow of blood within the limb 202 in correspondence with the withdrawal resistance being greater than (or equal to) the resistance threshold. In another example, the controller 102 may operate the force generator 300 when a rate of change of the withdrawal resistance exceeds the resistance threshold.

The controller 102 may cooperate with the cuff 200 to maintain vascular access resistance during blood filtration therapy with the system 100. For example, ultrafiltration (UF) removes one or more blood constituents from blood in the blood circuit 120. The access resistance may increase in correspondence with removal of plasma water from the circulatory system of the patient. In another example, vasoconstriction of the vasculature reduces volume space between the inner surface of the vasculature and outer surface of the catheter inserted into the vasculature. The reduction in volume space between the catheter and vasculature may correspondingly increase access resistance. The controller 102 may operate the force generator 300 to decrease the access resistance by driving blood flow from the vasculature and into the withdrawal line 104.

The blood circuit 120 (including the lines 104, 106) may have a total resistance characteristic that corresponds to an amount of resistance to the flow of blood in the blood circuit 120, for instance one or more of the withdrawal lines 104, infusion line 106, or the filter 110. One or more characteristics may contribute to the total resistance characteristic of the lines 104, 106. For example, the resistance characteristic of the lines 104, 106 may increase due to occlusion (e.g., clotting, obstruction, or the like) of the blood circuit 120 (e.g., in or around the catheter 108), changes to the vasculature (e.g., inflammation of walls of the vasculature, compression of the vasculature, or the like), hemoconcentration of the blood, or the like. The resistance characteristic (e.g., transverse or longitudinal) of the filter 110 may contribute to the total resistance characteristic of the blood circuit 120. As described herein, the controller 102 may operate the cuff 200 (e.g., force applied by the cuff 200) to inhibit (or permit) flow in the limb 202 of a patient based on access resistance of the patient (or the system 100).

Referring to FIG. 3, the controller 102 may operate the cuff 200 based on oxygen saturation of a patient. For example, the controller 102 may include an oxygen saturation sensor 308 (e.g., a pulse oximeter, or the like). In some examples, inhibiting arterial inflow reduces the oxygen saturation of the patient. According, the system 100 may monitor the oxygen saturation of the patient to maintain arterial inflow into the limb 202 of the patient. The sensor 308 may measure oxygen saturation of the patient. The controller 102 may monitor the sensor 308, and the controller 102 may monitor the oxygen saturation of the patient. The controller 102 may operate the cuff 200 based on the measured oxygen saturation of the patient. For instance, the controller 102 may vary force applied by the cuff 200 to the limb 202 when the oxygen saturation of the patient exceeds an oxygen saturation threshold. In one example, the controller may decrease the force applied to the limb (and permit blood flow) when measured oxygen saturation is less than 95 percent.

In another example, the controller 102 may use the cuff 200 to determine systolic arterial blood pressure. For instance, the controller 102 may limit the pressure applied to the limb to the determined systolic arterial blood pressure. In an example, the controller 102 may limit the pressure applied to the limb to be less than or equal to the determined systolic arterial blood pressure. In another example, the controller 102 may use the cuff 200 to determine diastolic pressure of the patient, or the like. For instance, the controller 102 may communicate with the cuff characteristic sensor 210 to determine pressure in vasculature of the patient. In yet another example, the system 100 uses the cuff characteristic sensor 210 to determine one or more of venous pressure, arterial pressure (e.g., systolic, diastolic, or the like), or the like. Accordingly, the system 100 may use the cuff 200 as a sphygmomanometer.

The system 100 may operate the cuff 200 based on the determined vascular pressure. For instance, the controller 102 may limit the pressure applied to the limb 202 in correspondence with inhibiting blood flow in the limb 202. In an example, the cuff 200 may apply a pressure greater than the determined vascular pressure to inhibit blood flow in the limb 202. The system 100 may monitor blood flow in the limb 202, for example with an ultrasound transducer or the like. The system 100 may reduce the pressure applied by the cuff 200 (e.g., to enhance patient comfort, or the like). For instance, the system 100 may reduce the pressure applied by the cuff 200 in correspondence with monitoring the blood flow in the limb 202. Thus, the system may inhibit blood flow in the limb 202 while limiting the pressure applied to the limb 202 (e.g., to enhance patient comfort, or the like.

Referring to FIG. 3, the controller 102 may include a sensor interface 310. The controller 102 may use the sensor interface 310 to communicate with the sensors 124. For instance, the sensors 124 may communicate the determined physiological parameters to the controller 102 using the sensor interface 310. Accordingly, the system 100 may monitor the physiological parameters of a patient using one or more of the controller 102 and the sensors 124.

In another example, the controller 102 may include a cuff module 312. The cuff module 312 may cooperate with one or more of the cuff 200 or the force generator 300 to operate the cuff 200. For instance, the system 100 may use the cuff module 312 to tighten (or loosen) the cuff. In another example, the system 100 uses the cuff module 312 to operate the cuff in correspondence with physiological characteristics of the patient (e.g., physiological characteristics determined with the sensors 124, or the like). For example, cuff module 312 may tighten or loosen the cuff based on vasculature pressure. The cuff module 312 may operate the cuff based on a change in the physiological characteristics of the patient. For instance, the cuff module 312 may operate the cuff 200 based on a change in blood flow rate in a vein (e.g., the first vein 205A, shown in FIG. 2) of a patient. Accordingly, in some examples, the system 100 may operate the cuff 200 to drive blood into the blood circuit 120 and improve performance of the blood filtration system 100.

The controller 102 may include a comparator 314. In an example, the controller 102 uses the comparator 102 to operate the cuff 200 (e.g., in cooperation with the cuff module 312, or the like). For instance, the comparator 314 may compare determined physiological parameters to a physiological threshold. The comparator 314 may compare vascular pressure of a patient to a pressure threshold. The cuff module 312 may operate the cuff 200 based on the comparison of the vascular pressure to the pressure threshold. In another example, the comparator 314 may compare access resistance of the system 100 to a resistance threshold. The cuff module 312 may operate the cuff 200 based on the comparison of the access resistance of the system 100 to the resistance threshold. In yet another example, the comparator 314 compares the venous flow in the limb 202 (e.g., determined using the sensor 302, or the like) to a venous flow threshold. The cuff module 312 may operate the cuff 200 based on the comparison of the venous flow to the venous flow threshold. Accordingly, the controller 102 may operate the cuff 200 to (selectively) inhibit blood flow in a limb of a patient in correspondence with physiological characteristics of the patient.

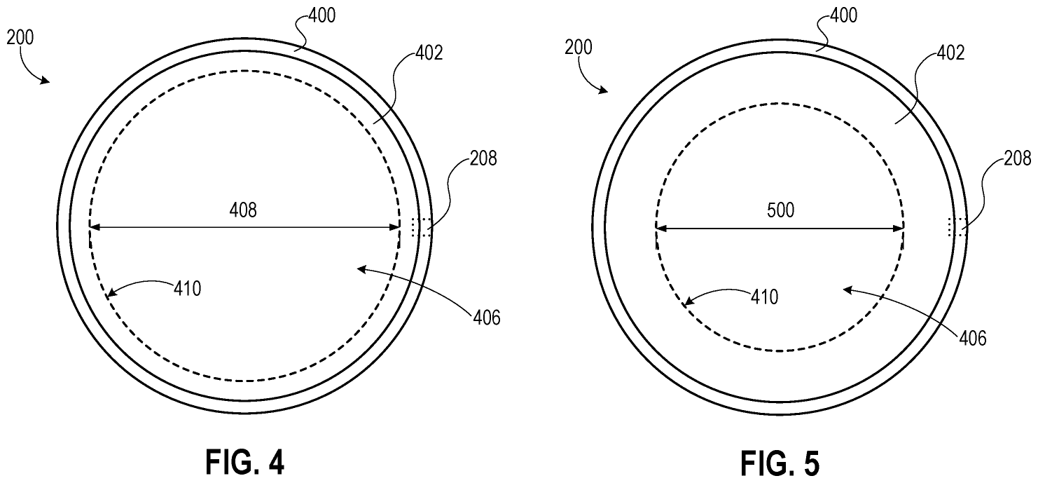

FIG. 4 shows the cuff 200 in a loosened configuration, and FIG. 5 shows the cuff 200 in a tightened configuration. The cuff 200 may include a shell 400, and the shell 400 may be rigid (e.g., hard, inflexible, or the like). The cuff 200 may include a bladder 402, and the bladder 402 may displace relative to the shell 400. For instance, the bladder 402 may expand to tighten the cuff 200. The bladder 402 may contract to loosen the cuff 200. The bladder 402 may inflate to tighten the cuff 200. The bladder 402 may deflate to loosen the cuff 200. In an example, a fluid (e.g., gas, liquid, or the like) may be pumped into (or out of) the bladder 402 through the conduit 208. The force generator 300 (e.g., pump 301A, shown in FIG. 3) may be in communication with the bladder 402 to pump fluid into (or out of) the bladder 402.

The cuff 200 may include a limb socket 406, and the size of the limb socket 406 may vary according to a change in the bladder 402 (e.g., a change between loosened and tightened configurations). For example, the limb socket 406 may have a first size 408 (shown in FIG. 4) with the cuff 200 in the loosened configuration. The bladder 402 may be tightened (e.g., by expanding the bladder 402, or the like), and the bladder 402 may displace relative to the shell 400 (and displace into the limb socket 406). Accordingly, the limb socket 406 may have a second size 500 (shown in FIG. 5) with the cuff in the tightened configuration. The second size 500 may be smaller than the first size 408 (shown in FIG. 4).

The cuff 200 may include a contact surface 410. For instance, the bladder 402 may include the contact surface 410. The contact surface 410 may engage with the limb, for example when the limb is received in the limb socket 406. Accordingly, the contact surface 410 may apply force (or pressure) to the limb received in the limb socket 406.

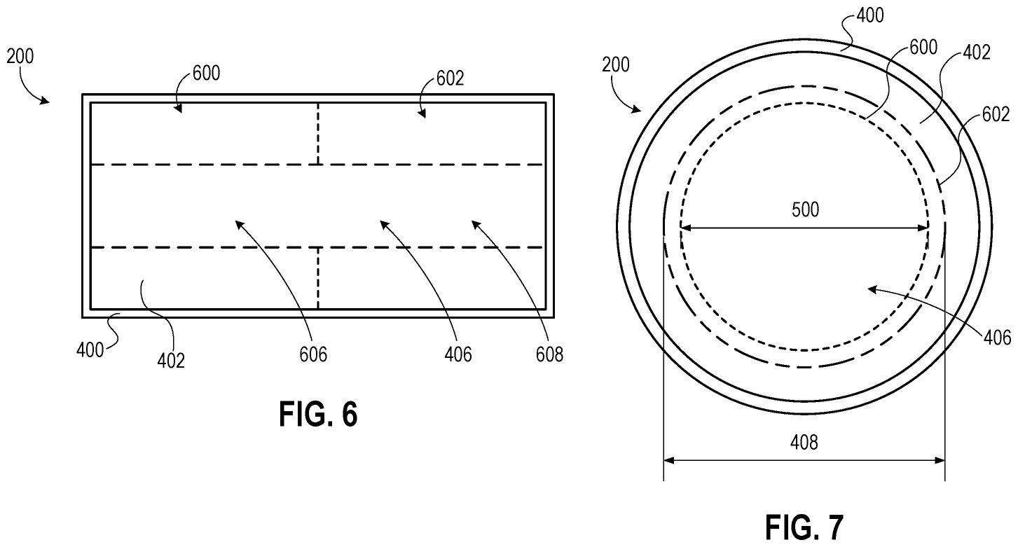

FIG. 6 shows a side view of the cuff 200 optionally including the bladder 402. The bladder 402 may include a first bladder section 600 and a second bladder section 602. The bladder sections 600, 602 may displace relative to the shell 400. For instance, the bladder section 600 is isolated from the bladder section 602. In an example, the bladder section 600 is separately adjustable relative to the bladder section 602. For example, the bladder section 600 may be inflated to apply a first pressure to a limb received in a first section 606 of the limb socket 406. The bladder section 600 may be inflated to apply a second pressure to the limb received in a second section 608 of the limb socket 406. The first pressure is optionally different than the first pressure. Thus, the cuff 200 may apply varying amounts of force (or pressure) to a limb received in the limb socket 406.

FIG. 7 shows an end view of the cuff 200. As described herein, the limb socket 406 may vary in size according to tightening (or loosening) of the bladder 402. The bladder sections 600, 602 may have different sizes. For instance, the bladder section 600 may have the second size 500, and the bladder section 602 may have the first size 408. Thus, the first section 606 (shown in FIG. 6) of the limb socket 406 may have the first size, and the second section 608 of the limb socket 406 may have the second size. The controller 102 may operate the cuff 200 to vary the size of the limb socket 406 (or the size of the sections 606, 608 of the limb socket 406).

In another example, the cuff 200 may be a first cuff having a first size (e.g., sized and shaped for attachment to a limb of a child). The cuff 200 may be a second cuff having a second size (e.g., sized and shaped for attachment to a limb of an adult). One or more of the cuff 200 or the bladder 402 may be cylindrical in shape, conical to match contour of the limb 202, or customizable to mimic anatomy of a specific patient. In yet another example, the cuff 200 may be included in a plurality of cuffs. The plurality of cuffs may have varying profiles (e.g., one or more of cross-section, shape, size, dimensions, contour, radius, perimeter, circumference, diameter, outline, boundary, configuration, pattern, arrangement, thickness, or the like). Accordingly, a physician may select among the plurality of cuffs to meet patient-specific needs. In an approach, cuff 200 may have a shape that does not match contours of the limb 202. Thus, a gap may exist at an interface between the cuff 200 and the limb 202. The gap may allow venous blood to flow in the vasculature. The gap may correspondingly result in an increase in pressure to inhibit flow within vasculature. Accordingly, a cuff 200 profile that matches limb contours may minimize pressure needed to inhibit blood flow in the limb (e.g., venous return, or the like). Thus, the cuff 200 may allow arterial inflow and minimize patient discomfort.

In yet another example, a first vein (e.g., vein 205A, shown in FIG. 2) is superficial and a second vein (e.g., vein 205B, shown in FIG. 2) is located deeper in the limb than the first vein. The force to inhibit flow in the second vein may be greater than the force to inhibit flow in the first vein. For instance, the cuff 200 may apply a first amount of force to a brachial vein of a patient. The cuff may apply a second amount of force to a basilic (or cephalic vein) in the upper arm. The first amount of force to inhibit flow in the brachial vein may be greater than the second amount of force to inhibit flow in the basilic vein. In an example, the controller 102 may operate the cuff 200 to apply the first amount of force or the second amount of force (e.g., to occlude superficial veins, or one or both of deep and superficial veins).

In still yet another example, and as described herein, the controller 102 may monitor venous flow of the patient (e.g., with the cuff deflated, or the like). The controller 102 may increase force applied by the cuff 200 (e.g., by operating the force generator 300, shown in FIG. 3) to inhibit (e.g., reduce, stop, occlude, slow, or the like) blood flow in vasculature of the patient. Accordingly, the cuff 200 may inhibit flow in the limb, for instance by inhibiting flow in a perforating vein (e.g., veins providing a flow path between superficial and deep veins). Thus, the cuff 200 may inhibit blood flow in the limb by applying force to the limb (and veins contained therein).

In a further example, a fluid is pumped into (or out of) the cuff 200 (e.g., with pump 201A, shown in FIG. 2). Gases are compressible, and liquids are incompressible (in comparison to gases). The cuff 200 may be filled (or evacuated) with a liquid, for example to enhance accuracy (or precision) of the application of force to the limb. For instance, filling the cuff (e.g., the bladder 402, shown in FIG. 4) with liquid may provide a linear application of force to the limb 202. The amount of force applied to a limb with the bladder 402 may vary according to the location of the cuff about the limb. Filling the bladder 402 with liquid reduces variations in force applied by the cuff due to the location of the cuff about the limb.

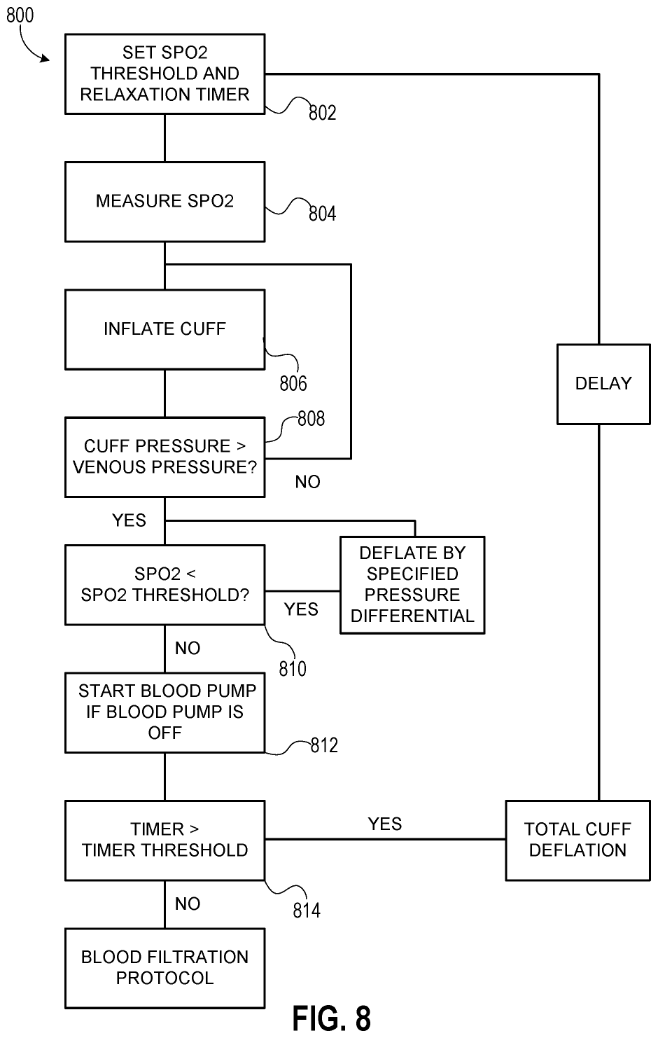

FIG. 8 shows an example of a method 800 for operating a cuff configured to inhibit blood flow in a limb of a patient. At 802, the method 800 includes setting one or more of an oxygen saturation threshold or a relaxation timer. The method 800 includes at 804 measuring the oxygen saturation of a patient (e.g., with sensor 308, shown in FIG. 3).

At 806, the cuff 200 may be tightened, for example by inflating the bladder 402. At 808, the controller 102 may compare the cuff pressure (e.g., provided by sensor 210, shown in FIG. 2) with venous pressure of the patient (e.g., provided by one or more of the sensors 124). In an example, the cuff is tightened until the cuff pressure is greater than (or equal to) the measured venous pressure of the patient.

The method 800 may include at 810 comparing the measured oxygen saturation to the oxygen saturation threshold. In an example, the cuff 200 is loosened if the measured oxygen saturation is less than the oxygen saturation threshold. At 812, the blood pump may be operated, for instance to pump blood through the blood circuit 120. At 814, the controller 102 may monitor the duration of time the cuff is tightened. The controller may compare the monitored duration that the cuff is tightened to the relaxation timer. The controller 102 may operate the cuff 200 to loosen the cuff when the monitored duration is greater than the relaxation timer.

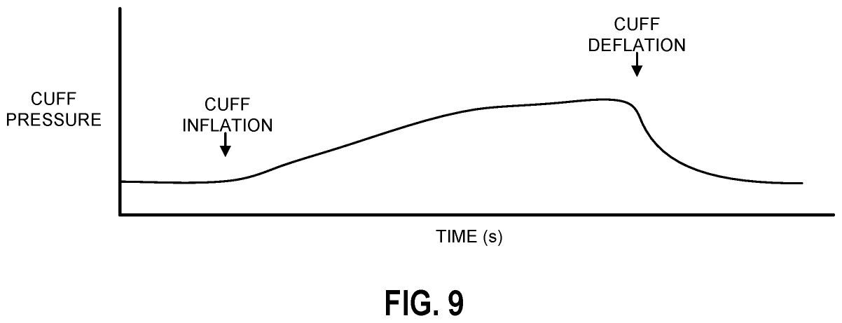

FIG. 9 shows a graphical representation of venous pressure in a limb with respect to time. In an example, the pressure shown in FIG. 9 corresponds to pressure distal to the cuff 200 (shown in FIG. 2). The cuff 200 may be tightened (e.g., inflated, constricted, expanded, closed, or the like). The pressure in the limb may increase when the cuff 200 is tightened. For instance, the cuff 200 may allow arterial inflow while inhibiting venous return. Accordingly, venous pressure in the limb 202 (shown in FIG. 2) distal to the cuff 200 may increase. The pressure in the limb may build. In an example, the pressure in the limb approaches a horizontal asymptote. Thus, pressure in the limb may approach a limit when the cuff 200 is tightened. The cuff 200 may be loosened (e.g., deflated, released, opened, contracted, or the like). In an example, loosening the cuff 200 may reduce pressure in the limb. For instance, loosening the cuff 200 may allow venous return, and pressure distal to the cuff may decrease as blood flows proximally (e.g., blood may flow past the cuff 200 with the cuff 200 loosened).

FIG. 10 shows an example of the cuff 200 attached to a leg 1000 of a patient. As described herein, the cuff 200 may be located proximate a joint of the limb 202. In an example, the cuff 200 may be located proximate a knee of the leg 1000. For instance, the cuff 200 may be attached to the leg 1000 proximate a popliteal fossa of the leg 1000. The cuff 200 may be attached to a thigh of a patient. The cuff 200 may be located proximate an ankle of the limb 202. In another example, the catheter 204 (associated with withdrawal line 104) may communicate with vasculature distal to the cuff 200 (e.g., a vein in an ankle or calf of a patient, or the like). The catheter 206 (associated with infusion line 106) may communicate with vasculature proximal to the cuff 200 (e.g., a vein in the thigh or buttock of a patient, or the like).

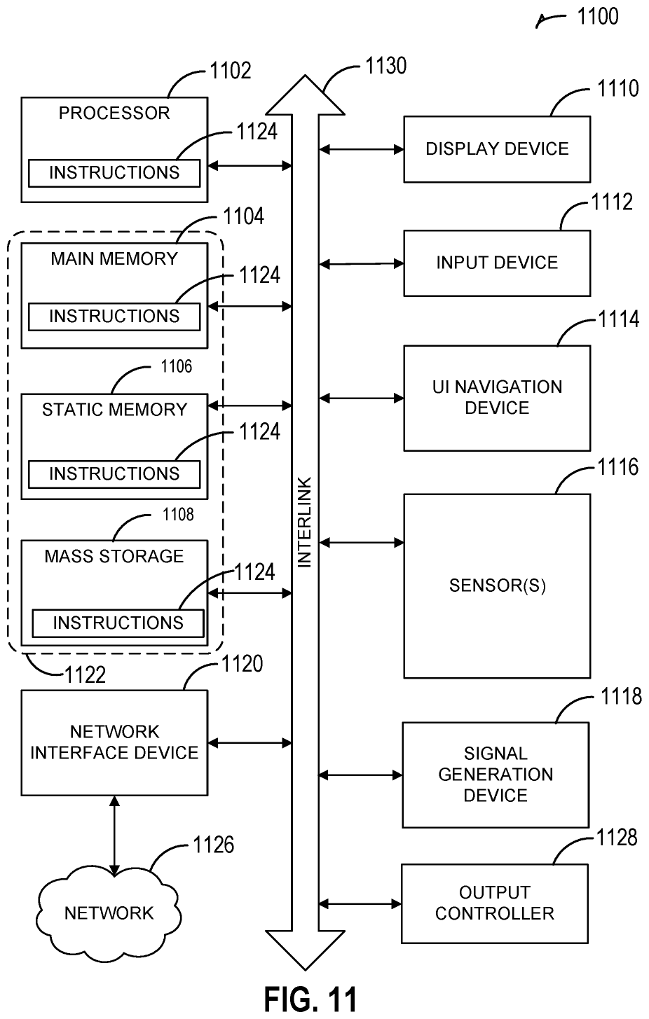

FIG. 11 shows a block diagram of an example machine 1100 upon which any one or more of the techniques (e.g., methodologies) discussed herein may perform. Examples, as described herein, may include, or may operate by, logic or a number of components, or mechanisms in the machine 1100. Circuitry (e.g., processing circuitry) is a collection of circuits implemented in tangible entities of the machine 1100 that include hardware (e.g., simple circuits, gates, logic, etc.), for instance the controller 102 (shown in FIG. 1). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation.

In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1100 may include a hardware processor 1102 (e.g., a central processing unit (CPU), a graphics processing unit (GPU), a hardware processor core, or any combination thereof), a main memory 1104, a static memory (e.g., memory or storage for firmware, microcode, a basic-input-output (BIOS), unified extensible firmware interface (UEFI), etc.) 1106, and mass storage 1108 (e.g., hard drive, tape drive, flash storage, or other block devices) some or all of which may communicate with each other via an interlink (e.g., bus) 1130. The machine 1100 may further include a display unit 1110, an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device (e.g., drive unit) 1108, a signal generation device 1118 (e.g., a speaker), a network interface device 1120, and one or more sensors 1116, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine readable media 1122. While the machine readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software. A transmission medium is a machine readable medium.

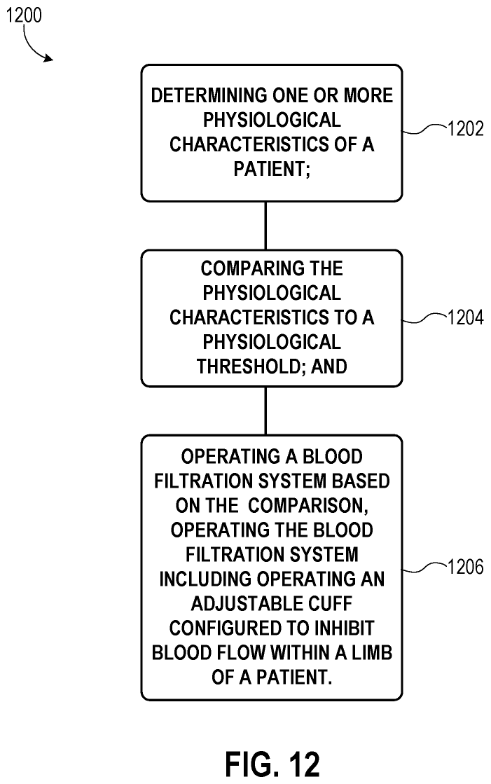

FIG. 12 shows one example of a method 1200 for operating a blood filtration system, including one or more of the blood filtration system 100, the controller 102, the cuff 200, or the force generator 300 described herein. In describing the method 1200, reference is made to one or more components, features, functions and operations previously described herein. Where convenient, reference is made to the components, features, operations and the like with reference numerals. The reference numerals provided are exemplary and are not exclusive. For instance, components, features, functions, operations and the like described in the method 1200 include, but are not limited to, the corresponding numbered elements provided herein and other corresponding elements described herein (both numbered and unnumbered) as well as their equivalents.

In an example, at 1202 the method 1200 may include determining one or more physiological characteristics of a patient. For example, the system 100 may determine physiological characteristics of a patient using one or more of the sensors 124. The sensors 124 may communicate the determined physiological characteristics with the controller 102. For instance, the sensor interface 310 may receive the physiological characteristics determined by the sensors 124.

In another example, the method 1200 may include at 1204 comparing the physiological characteristics to a physiological threshold. For example, the system 100 may use the comparator 314 to compare physiological characteristics of the patient to a physiological threshold. In an example, the sensors 124 may determine the flow rate of blood in vasculature of a patient. The comparator 314 may compare the flow rate of blood in vasculature of the patient to a flow rate threshold. In another example, the comparator 314 may compare the physiological characteristics to characteristics of the blood filtration system 100. For instance, the sensors 124 may determine pressure in vasculature of a patient. The comparator 314 may compare the pressure in the vasculature of the patient to a cuff pressure (e.g., a pressure within a bladder of the cuff 200, a pressure applied to the limb with the cuff 200, or the like).

In yet another example, the comparator 314 may compare characteristics of the blood filtration system 100. For instance, the comparator 314 may compare the cuff pressure to a cuff pressure threshold. In still yet another example, the comparator 314 may compare one or more of physiological characteristics of a patient or characteristics of the system 100 to a user-input. For instance, a user (e.g., healthcare provider, technician, or the like) may determine a vasculature pressure (e.g., venous, systolic, diastolic, or the like) of a patient. The system 100 may use the comparator 314 to compare the pressure applied by the cuff 200 to a vasculature pressure of a patient. Accordingly, the blood filtration system 100 may operate the cuff 200 to inhibit flow of blood within a limb of a patient.

In a further example, at 120 the method 1200 may include operating the blood filtration system based on the comparison. For instance, the system 100 may use the cuff module 312 to operate the cuff 200 and inhibit blood flow in a limb of a patient. The cuff module 312 may operate the cuff 200 based on a comparison of physiological characteristics to a physiological threshold (e.g., a specified blood flow rate in a limb of a patient, or the like). In another example, the cuff module 312 may operate the cuff 200 based on a comparison of system characteristics to a system threshold (e.g., a specified pressure applied by the cuff 200 to the limb, or the like). The cuff module 312 may operate the cuff 200 based on a comparison of physiological characteristics to a system characteristics (e.g., comparing pressure within vasculature of a limb to the pressure applied by the cuff 200 to the limb, or the like). Accordingly, the blood filtration system 100 may use one or more of physiological characteristics of the patient or characteristics of the system 100 to operate components of the system 100 including (but not limited to) the cuff 200, force generator 300, blood pump 112, or the like.

Various Notes & Aspects

Example 1 is a blood filtration system, comprising: a blood circuit including a withdrawal line, the withdrawal line configured to transmit blood from vasculature of a patient; an adjustable cuff configured to receive a portion of a limb of a patient, wherein: the cuff is configured to selectively engage with the limb to apply an external force to the limb and correspondingly inhibit flow of blood within the vasculature of the limb; and the cuff is adjustable to change the force applied to the limb and correspondingly change flow of blood from the vasculature to the blood circuit; and a controller in communication with the cuff and configured to operate the cuff to adjust the force applied to the limb of the patient.

In Example 2, the subject matter of Example 1 optionally includes wherein the cuff is configured to inhibit distal blood flow in the vasculature and correspondingly redirect the blood flow into the withdrawal line.

In Example 3, the subject matter of any one or more of Examples 1-2 optionally include wherein the cuff is configured to inhibit venous return within the limb.

In Example 4, the subject matter of any one or more of Examples 1-3 optionally include wherein: the cuff is configured to inhibit venous return within the limb; and the cuff is configured to permit arterial inflow into the limb.

In Example 5, the subject matter of any one or more of Examples 1˜4 optionally include wherein: the blood filtration system includes one or more physiological sensors configured to determine a venous flow rate of the patient; the controller is configured to monitor the venous flow rate using the physiological sensors; and the controller is configured to operate the cuff to adjust the force applied to the limb in correspondence with the monitored venous flow rate.

In Example 6, the subject matter of Example 5 optionally includes wherein: the controller is configured to compare the monitored venous flow rate with a venous flow rate threshold; and the controller is configured to operate the cuff to adjust the force applied to the limb based on the comparison of the monitored venous flow rate with the venous flow rate threshold.

In Example 7, the subject matter of any one or more of Examples 5-6 optionally include wherein the withdrawal line is configured for communication with a first portion of the vasculature and the physiological sensors are configured to determine the venous flow rate of a second portion of the vasculature.

In Example 8, the subject matter of any one or more of Examples 1-7 optionally include wherein: the blood filtration system includes one or more physiological sensors configured to determine a venous flow rate of the patient; the controller is configured to monitor the venous flow rate using the physiological sensors; and the controller is configured to operate the cuff to adjust the force applied to the limb based on changes in the monitored venous flow rate.

In Example 9, the subject matter of Example 8 optionally includes wherein the controller is configured to operate the cuff to adjust the force applied to the limb based on a decrease in the monitored venous flow rate.

In Example 10, the subject matter of Example 9 optionally includes wherein the controller is configured to operate the cuff and increase the force applied to the limb based on the decrease in the monitored venous flow rate.

In Example 11, the subject matter of any one or more of Examples 8-10 optionally include wherein the controller is configured to operate the cuff to adjust the force applied to the limb based on an increase in the monitored venous flow rate.

In Example 12, the subject matter of Example 11 optionally includes wherein the controller is configured to operate the cuff and decrease the force applied to the limb based on the increase in the monitored venous flow rate.

In Example 13, the subject matter of any one or more of Examples 1-12 optionally include wherein: the blood filtration system includes one or more physiological sensors configured to determine a venous pressure of the patient; the controller is in communication with the one or more physiological sensors; and the controller is configured to operate the cuff to adjust the force applied to the limb in correspondence with the determined venous pressure.

In Example 14, the subject matter of Example 13 optionally includes wherein the controller is configured to adjust a cuff pressure corresponding to the force applied to the limb such that the cuff pressure is greater than or equal to the venous pressure.

In Example 15, the subject matter of any one or more of Examples 1-14 optionally include wherein the controller is configured to operate the cuff to allow arterial inflow into the limb in correspondence with inhibiting venous return from the limb.

In Example 16, the subject matter of Example 15 optionally includes one or more physiological sensors configured to determine one or more of a venous pressure or an arterial pressure of the patient, wherein the controller is configured to adjust the force applied to the limb to correspondingly apply a pressure that is greater than or equal to the venous pressure.

In Example 17, the subject matter of Example 16 optionally includes wherein the controller is configured to adjust the force applied to the limb to correspondingly apply a pressure that is less than or equal to one or more of systolic arterial pressure or diastolic arterial pressure.

In Example 18, the subject matter of any one or more of Examples 1-17 optionally include wherein the limb of the patient includes an arm of the patient.

In Example 19, the subject matter of any one or more of Examples 1-18 optionally include wherein the limb of the patient includes a leg of the patient.

Example 20 is a blood filtration system, comprising: one or more sensors configured to determine physiological parameters of a patient; a cuff pump configured for communication with an adjustable cuff, wherein the adjustable cuff is configured to receive a fluid to inflate or deflate the adjustable cuff, and the cuff pump is configured to supply the fluid to the adjustable cuff; a controller including: a cuff module configured to operate the cuff pump; a sensor interface configured to communicate with the one or more sensors; a comparator configured to compare the physiological parameters of the patient to a physiological threshold; and wherein the cuff module is configured to operate the cuff pump based on the comparison.

In Example 21, the subject matter of Example 20 optionally includes the cuff

In Example 22, the subject matter of any one or more of Examples 20-21 optionally include wherein the one or more sensors include a pulse oximeter configured to determine oxygen saturation of a patient.

In Example 23, the subject matter of Example 22 optionally includes wherein the pump module is configured to operate the cuff pump based on a comparison of the oxygen saturation of the patient to an oxygen saturation threshold.

In Example 24, the subject matter of any one or more of Examples 22-23 optionally include wherein the pulse oximeter is configured for attachment to a patient distally with respect to the adjustable cuff.

In Example 25, the subject matter of any one or more of Examples 20-24 optionally include wherein the one or more physiological sensors include an ultrasonic transducer configured to determine flow rate of blood in vasculature of the patient.

In Example 26, the subject matter of Example 25 optionally includes wherein the pump module is configured to operate the cuff pump based on a comparison of the flow rate of blood to a flow rate threshold.

In Example 27, the subject matter of any one or more of Examples 20-26 optionally include wherein the one or more physiological sensors are configured to determine one or more of force or pressure supplied to the adjustable cuff by the cuff pump.

In Example 28, the subject matter of any one or more of Examples 20-27 optionally include wherein: the physiological parameters include one or more of venous pressure or arterial pressure of a patient; the comparator is configured to compare the one or more venous pressure or arterial pressure to a pressure threshold; and the cuff pump is configured to operate the cuff based on the comparison of the one or more venous pressure or arterial pressure to the pressure threshold.

In Example 29, the subject matter of Example 28 optionally includes wherein the cuff pump is configured to adjust the force applied to the limb to correspondingly apply a pressure that is greater than or equal to the venous pressure.

In Example 30, the subject matter of Example 29 optionally includes wherein the cuff pump is configured to adjust the force applied to the limb to correspondingly apply a pressure that is less than or equal to the arterial pressure.

Example 31 is a blood filtration system, comprising: a withdrawal line and an infusion line configured to transmit blood from vasculature, wherein the withdrawal line and the infusion line are configured to couple with a filter, and the filter is configured to remove one or more plasma constituents from the blood of a patient; an adjustable cuff configured to receive a portion of a limb of a patient, wherein: the cuff is configured to engage with the limb to apply a force to the limb, and the engagement of the cuff with the limb is configured to inhibit flow of blood within the limb; and wherein the withdrawal line is configured to communicate with vasculature located distally with respect to the cuff, and the infusion line is configured to communicate with vasculature located proximally with respect to the cuff.

In Example 32, the subject matter of Example 31 optionally includes wherein the cuff is configured to inhibit venous return within the limb.

In Example 33, the subject matter of any one or more of Examples 31-32 optionally include wherein: the cuff is configured to inhibit venous return within the limb; and the cuff is configured to permit arterial inflow into the limb.

In Example 34, the subject matter of any one or more of Examples 31-33 optionally include wherein: the withdrawal line is configured to couple with a withdrawal catheter; the infusion line is configured to couple with an infusion catheter; and the withdrawal catheter is separate from the infusion catheter.

Example 35 is a blood filtration system, comprising: a withdrawal line and an infusion line configured to transmit blood from vasculature, wherein the withdrawal line and the infusion line are configured to couple with a filter, and the filter is configured to remove one or more plasma constituents from the blood of the patient; an adjustable cuff configured to receive a portion of a limb of a patient, wherein: the cuff is configured to engage with the limb to apply a force to the limb, and the engagement of the cuff with the limb is configured to inhibit flow of blood within the limb; and a controller, wherein: the controller is configured to modulate the cuff to adjust the force applied to the limb of the patient.

In Example 36, the subject matter of Example 35 optionally includes wherein the cuff is configured to inhibit venous return within the limb.

In Example 37, the subject matter of any one or more of Examples 35-36 optionally include wherein: the cuff is configured to inhibit venous return within the limb; and the cuff is configured to permit arterial inflow into the limb.

In Example 38, the subject matter of any one or more of Examples 35-37 optionally include wherein: the withdrawal line is configured to communicate with vasculature located distally with respect to the cuff.

In Example 39, the subject matter of any one or more of Examples 35-38 optionally include wherein: the infusion line is configured to communicate with vasculature located proximally with respect to the cuff.

In Example 40, the subject matter of any one or more of Examples 35-39 optionally include wherein the cuff is configured to engage with the limb of a patient proximate to an elbow of the patient.

In Example 41, the subject matter of Example 40 optionally includes wherein: the withdrawal line is configured to communicate with vasculature located distally with respect to the elbow; and the infusion line is configured to communicate with vasculature located proximal with respect to the elbow.

In Example 42, the subject matter of any one or more of Examples 35-41 optionally include wherein: the cuff includes an arm socket; and the cuff includes an initial configuration and a tightened configuration, wherein: in the initial configuration, the arm socket has a first size; in the tightened configuration, the arm socket has a second size; and the second size is smaller than the first size.

In Example 43, the subject matter of Example 42 optionally includes wherein the tightened configuration is a first tightened configuration, and wherein: the cuff includes a second tightened configuration, wherein in the second tightened configuration, the arm socket has a third size; and the third size is smaller than the second size.

In Example 44, the subject matter of any one or more of Examples 42-43 optionally include wherein the controller is configured to modulate the cuff to transition the cuff between the initial configuration and the tightened configuration.

In Example 45, the subject matter of any one or more of Examples 35-44 optionally include wherein the cuff is configured to constrict about the limb of the patient.

In Example 46, the subject matter of any one or more of Examples 35-45 optionally include wherein the cuff is configured to tighten about the limb and configured to loosen about the limb, wherein the force applied to the limb is varied based on tightening or loosening of the cuff about the limb.

In Example 47, the subject matter of any one or more of Examples 35-46 optionally include wherein: the cuff includes an arm socket; the cuff includes a deflated configuration and an inflated configuration, wherein: in the deflated configuration, the arm socket has a first size; in the inflated configuration, the arm socket has a second size; and the second size is smaller than the first size. In Example 48, the subject matter of any one or more of Examples 35-47 optionally include wherein: the withdrawal line is configured to couple with a withdrawal catheter; the infusion line is configured to couple with an infusion catheter; and the withdrawal catheter is separate from the infusion catheter.

In Example 49, the subject matter of Example 48 optionally includes wherein: the withdrawal catheter includes a first needle catheter; and the infusion catheter includes a second needle catheter.

In Example 50, the subject matter of any one or more of Examples 48-49 optionally include wherein: the withdrawal catheter includes a withdrawal lumen tip; the withdrawal lumen tip is configured for alignment against flow of blood within the vasculature; the infusion catheter includes an infusion lumen tip; and the infusion lumen tip is configured for alignment with flow of blood within the vasculature.

In Example 51, the subject matter of any one or more of Examples 48-50 optionally include wherein: the withdrawal catheter is configured for insertion into a first vein; the infusion catheter is configured for insertion into a second vein; and the first vein is different than the second vein.

In Example 52, the subject matter of Example 51 optionally includes wherein: the first vein is distal of a cubital fossa of a patient; and the second vein is proximal of a cubital fossa of a patient.

In Example 53, the subject matter of any one or more of Examples 35-52 optionally include wherein: the cuff is configured to inhibit flow within veins of the patient; and the cuff is configured to permit flow within arteries of the patient.

In Example 54, the subject matter of any one or more of Examples 35-53 optionally include wherein the cuff is configured to extend along a cubital fossa of a patient.

In Example 55, the subject matter of any one or more of Examples 35-54 optionally include wherein the cuff includes a guide configured to receive one or more of the withdrawal line or the infusion line.

In Example 56, the subject matter of any one or more of Examples 35-55 optionally include wherein the cuff includes a heating element configured to generate heat.

In Example 57, the subject matter of any one or more of Examples 35-56 optionally include wherein: the cuff includes a first bladder section and a second bladder section; the cuff includes a first arm socket section and a second arm socket section; and the first bladder section includes a first initial configuration and a first tightened configuration, wherein: in the first initial configuration, the first arm socket section has a first size; in the first tightened configuration, the first arm socket section has a second size; and the second size is smaller than the first size; the second bladder section includes a second initial configuration and a second tightened configuration, wherein: in the second initial configuration, the second arm socket section has a third size; in the second tightened configuration, the second arm socket section has a fourth size; and the fourth size is smaller than the third size.

In Example 58, the subject matter of Example 57 optionally includes wherein the second size is different than the first size.

In Example 59, the subject matter of any one or more of Examples 57-58 optionally include wherein the first size is different than the third size.

In Example 60, the subject matter of any one or more of Examples 35-59 optionally include wherein: the cuff includes a limb socket configured to receive the limb of the patient; the cuff includes a rigid shell surrounding the limb socket; the cuff includes a bladder coupled with the rigid shell; and the bladder has a contact surface configured to engage with the limb in the limb socket; the bladder is configured to displace relative to the rigid shell to vary a size of the limb socket.

In Example 61, the subject matter of Example 60 optionally includes wherein: the bladder includes an inflated configuration and a deflated configuration; in the inflated configuration, the limb socket has a first size; in the deflated configuration, the limb has a second size; and the first size is smaller than the second size.

In Example 62, the subject matter of any one or more of Examples 35-61 optionally include a sensor configured to measure one or more physiological characteristics of the patient, wherein: the controller is configured to modulate the cuff based on the measured physiological characteristics.

In Example 63, the subject matter of Example 62 optionally includes wherein the controller is configured to modulate the cuff to adjust the force applied to the limb of the patient in proportion to a change in the measured physiological characteristics.

In Example 64, the subject matter of any one or more of Examples 62-63 optionally include wherein: the sensor is configured to measure venous pressure of the patient; and the controller is configured to modulate the cuff based on the venous pressure of the patient.

In Example 65, the subject matter of Example 64 optionally includes wherein: the cuff is configured to apply a cuff pressure to the limb of the patient, the cuff pressure based on the force applied by the cuff; the controller is configured to modulate the cuff to vary the cuff pressure between a first cuff pressure and a second cuff pressure.

In Example 66, the subject matter of Example 65 optionally includes wherein the first cuff pressure is less than the venous pressure.

In Example 67, the subject matter of any one or more of Examples 65-66 optionally include wherein the second cuff pressure is greater than the venous pressure.

In Example 68, the subject matter of any one or more of Examples 65-67 optionally include wherein the second cuff pressure is greater than or equal to the venous pressure.

In Example 69, the subject matter of any one or more of Examples 64-68 optionally include wherein the sensor is configured to determine venous pressure in one or more of a cephalic vein or a brachial vein.

In Example 70, the subject matter of any one or more of Examples 62-69 optionally include wherein: the sensor is configured to measure venous flow of the patient; and the controller is configured to modulate the cuff based on the venous flow of the patient.

In Example 71, the subject matter of any one or more of Examples 62-70 optionally include wherein: the sensor is configured to measure oxygen saturation of the patient; and the controller is configured to modulate the cuff based on the oxygen saturation of the patient.

In Example 72, the subject matter of Example 71 optionally includes wherein: the controller is configured to compare the measured oxygen saturation to an oxygen saturation threshold; the controller is configured to vary force applied to the limb by the cuff when the measured oxygen saturation exceeds the oxygen saturation threshold.

In Example 73, the subject matter of Example 72 optionally includes wherein: the oxygen saturation threshold includes the oxygen saturation value of 95 percent; and the controller is configured to decrease force applied to the limb by the cuff when the measured oxygen saturation is less than or equal to the oxygen saturation threshold.

In Example 74, the subject matter of any one or more of Examples 62-73 optionally include wherein: the sensor is configured to measure respiration of the patient; and the controller is configured to modulate the cuff based on the respiration of the patient.

In Example 75, the subject matter of any one or more of Examples 62-74 optionally include wherein the sensor includes a Doppler velocimeter configured to measure blood flow of the patient.

In Example 76, the subject matter of any one or more of Examples 62-75 optionally include wherein: the sensor is configured to measure pressure in one or more of the withdrawal line or the infusion line; and the controller is configured to determine resistance in one or more of the withdrawal line or the infusion line based on the measured pressure.

In Example 77, the subject matter of Example 76 optionally includes wherein the controller is configured to modulate the cuff to increase force applied to the limb based on an increase in the flow resistance in one or more of the withdrawal line or the infusion line.

In Example 78, the subject matter of any one or more of Examples 62-77 optionally include wherein: the sensor is configured to measure venous pressure of the patient; the sensor is configured to measure oxygen saturation of the patient; and the controller is configured to modulate the cuff based on one or more of the venous pressure or the oxygen saturation of the patient.

In Example 79, the subject matter of any one or more of Examples 35-78 optionally include wherein controller is configured to modulate the cuff to apply a specified force for a specified time period.

In Example 80, the subject matter of any one or more of Examples 35-79 optionally include a variable-speed blood pump configured to pump blood in the withdrawal line, through the filter, and into the infusion line.

In Example 81, the subject matter of any one or more of Examples 35-80 optionally include a variable speed filtration pump configured to extract a filtrate fluid from the filter, wherein the filtrate fluid includes the one or more plasma constituents.

In Example 82, the subject matter of any one or more of Examples 35-81 optionally include wherein the cuff is configured to inhibit recirculation of blood from the infusion line to the withdrawal line.

Example 83 is a blood filtration system, comprising: an adjustable cuff configured to receive a portion of a limb of a patient, wherein: the cuff is configured to engage with the limb to apply a force to the limb, and the engagement of the cuff with the limb is configured to inhibit flow of blood within the limb; and a controller, wherein: the controller is configured to modulate the cuff to adjust the force applied to the limb of the patient.

In Example 84, the subject matter of Example 83 optionally includes wherein the cuff is configured to inhibit venous return within the limb.

In Example 85, the subject matter of any one or more of Examples 83-84 optionally include wherein: the cuff is configured to inhibit venous return within the limb; and the cuff is configured to permit arterial inflow into the limb.

In Example 86, the subject matter of any one or more of Examples 83-85 optionally include wherein the cuff is configured to engage with the limb of a patient proximate to an elbow of the patient.

In Example 87, the subject matter of any one or more of Examples 83-86 optionally include wherein: the cuff includes an arm socket; and the cuff includes an initial configuration and a tightened configuration, wherein: in the initial configuration, the arm socket has a first size; in the tightened configuration, the arm socket has a second size; and the second size is smaller than the first size.

In Example 88, the subject matter of Example 87 optionally includes wherein the tightened configuration is a first tightened configuration, and wherein: the cuff includes a second tightened configuration, wherein in the second tightened configuration, the arm socket has a third size; and the third size is smaller than the second size.

In Example 89, the subject matter of any one or more of Examples 87-88 optionally include wherein the controller is configured to modulate the cuff to transition the cuff between the initial configuration and the tightened configuration.

In Example 90, the subject matter of any one or more of Examples 83-89 optionally include wherein the cuff is configured to constrict about the limb of the patient.

In Example 91, the subject matter of any one or more of Examples 83-90 optionally include wherein the cuff is configured to tighten about the limb and configured to loosen about the limb, wherein the force applied to the limb is varied based on tightening or loosening of the cuff about the limb.

In Example 92, the subject matter of any one or more of Examples 83-91 optionally include wherein: the cuff includes an arm socket; the cuff includes a deflated configuration and an inflated configuration, wherein: in the deflated configuration, the arm socket has a first size; in the inflated configuration, the arm socket has a second size; and the second size is smaller than the first size.

In Example 93, the subject matter of any one or more of Examples 83-92 optionally include wherein the cuff includes a guide configured to receive one or more of the withdrawal line or the infusion line.

In Example 94, the subject matter of any one or more of Examples 83-93 optionally include wherein: the cuff includes a first bladder section and a second bladder section; the cuff includes a first arm socket section and a second arm socket section; and the first bladder section includes a first initial configuration and a first tightened configuration, wherein: in the first initial configuration, the first arm socket section has a first size; in the first tightened configuration, the first arm socket section has a second size; and the second size is smaller than the first size; the second bladder section includes a second initial configuration and a second tightened configuration, wherein: in the second initial configuration, the second arm socket section has a third size; in the second tightened configuration, the second arm socket section has a fourth size; and the fourth size is smaller than the third size.

In Example 95, the subject matter of Example 94 optionally includes wherein the second size is different than the first size.

In Example 96, the subject matter of any one or more of Examples 94-95 optionally include wherein the first size is different than the third size.

In Example 97, the subject matter of any one or more of Examples 83-96 optionally include wherein: the cuff includes a limb socket configured to receive the limb of the patient; the cuff includes a rigid shell surrounding the limb socket; the cuff includes a bladder coupled with the rigid shell; and the bladder has a contact surface configured to engage with the limb in the limb socket; the bladder is configured to displace relative to the rigid shell to vary a size of the limb socket.

In Example 98, the subject matter of Example 97 optionally includes wherein: the bladder includes an inflated configuration and a deflated configuration; in the inflated configuration, the limb socket has a first size; in the deflated configuration, the limb has a second size; and the first size is smaller than the second size.

In Example 99, the subject matter of any one or more of Examples 83-98 optionally include a sensor configured to measure one or more physiological characteristics of the patient, wherein: the controller is configured to modulate the cuff based on the measured physiological characteristics.

In Example 100, the subject matter of Example 99 optionally includes wherein the controller is configured to modulate the cuff to adjust the force applied to the limb of the patient in proportion to a change in the measured physiological characteristics.

In Example 101, the subject matter of any one or more of Examples 99-100 optionally include wherein: the sensor is configured to measure venous pressure of the patient; and the controller is configured to modulate the cuff based on the venous pressure of the patient.

In Example 102, the subject matter of Example 101 optionally includes wherein: the cuff is configured to apply a cuff pressure to the limb of the patient, the cuff pressure based on the force applied by the cuff; the controller is configured to modulate the cuff to vary the cuff pressure between a first cuff pressure and a second cuff pressure.

In Example 103, the subject matter of Example 102 optionally includes wherein the first cuff pressure is less than the venous pressure.

In Example 104, the subject matter of any one or more of Examples 102-103 optionally include wherein the second cuff pressure is greater than the venous pressure.

In Example 105, the subject matter of any one or more of Examples 102-104 optionally include wherein the second cuff pressure is greater than or equal to the venous pressure.

In Example 106, the subject matter of any one or more of Examples 101-105 optionally include wherein the sensor is configured to determine venous pressure in one or more of a cephalic vein or a brachial vein.

In Example 107, the subject matter of any one or more of Examples 99-106 optionally include wherein: the sensor is configured to measure venous flow of the patient; and the controller is configured to modulate the cuff based on the venous flow of the patient.

In Example 108, the subject matter of any one or more of Examples 99-107 optionally include wherein: the sensor is configured to measure oxygen saturation of the patient; and the controller is configured to modulate the cuff based on the oxygen saturation of the patient.

In Example 109, the subject matter of Example 108 optionally includes wherein: the controller is configured to compare the measured oxygen saturation to an oxygen saturation threshold; the controller is configured to vary force applied to the limb by the cuff when the measured oxygen saturation exceeds the oxygen saturation threshold.

In Example 110, the subject matter of Example 109 optionally includes wherein: the oxygen saturation threshold includes the oxygen saturation value of 95 percent; and the controller is configured to decrease force applied to the limb by the cuff when the measured oxygen saturation is less than or equal to the oxygen saturation threshold.

In Example 111, the subject matter of any one or more of Examples 99-110 optionally include wherein: the sensor is configured to measure respiration of the patient; and the controller is configured to modulate the cuff based on the respiration of the patient.

In Example 112, the subject matter of any one or more of Examples 99-111 optionally include wherein the sensor includes a Doppler velocimeter configured to measure blood flow of the patient.

In Example 113, the subject matter of any one or more of Examples 99-112 optionally include wherein: the sensor is configured to measure pressure in one or more of the withdrawal line or the infusion line; and the controller is configured to determine resistance in one or more of the withdrawal line or the infusion line based on the measured pressure.

In Example 114, the subject matter of Example 113 optionally includes wherein the controller is configured to modulate the cuff to increase force applied to the limb based on an increase in the flow resistance in one or more of the withdrawal line or the infusion line.

In Example 115, the subject matter of any one or more of Examples 99-114 optionally include wherein: the sensor is configured to measure venous pressure of the patient; the sensor is configured to measure oxygen saturation of the patient; and the controller is configured to modulate the cuff based on one or more of the venous pressure or the oxygen saturation of the patient.

In Example 116, the subject matter of any one or more of Examples 83-115 optionally include wherein controller is configured to modulate the cuff to apply a specified force for a specified time period.

In Example 117, the subject matter of any one or more of Examples 83-116 optionally include wherein the cuff is configured to inhibit recirculation of blood from the infusion line to the withdrawal line.

Each of these non-limiting examples may stand on its own, or may be combined in various permutations or combinations with one or more of the other examples.

The above description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A blood filtration system, comprising:
a blood circuit including a withdrawal line, the withdrawal line configured to transmit blood from vasculature of a patient;
an adjustable cuff configured to receive a portion of a limb of a patient, wherein:
the cuff is configured to selectively engage with the limb to apply an external force to the limb and correspondingly inhibit flow of blood within the vasculature of the limb; and
the cuff is adjustable to change the force applied to the limb and correspondingly change flow of blood from the vasculature the blood circuit; and
a controller in communication with the cuff and configured to operate the cuff to adjust the force applied to the limb of the patient, wherein:
the controller is configured to monitor venous flow rate using one or more physiological sensors; and
the controller is configured to operate the cuff to adjust the force applied to the limb based on changes in the monitored venous flow rate, including:
the controller is configured to operate the cuff and increase the force applied to the limb based on a decrease in the monitored venous flow rate; or
the controller is configured to operate the cuff and decrease the force applied to the limb based on an increase in the monitored venous flow rate.

2. The blood filtration system of claim 1, wherein the cuff is configured to inhibit distal blood flow in the vasculature and correspondingly redirect the blood flow into the withdrawal line.

3. The blood filtration system of claim 1, wherein the cuff is configured to inhibit venous return within the limb.

4. The blood filtration system of claim 1, wherein:
the cuff is configured to inhibit venous return within the limb; and
the cuff is configured to permit arterial inflow into the limb.

5. (canceled)

6. The blood filtration system of claim 1, wherein:
the controller is configured to compare the monitored venous flow rate with a venous flow rate threshold; and
the controller is configured to operate the cuff to adjust the force applied to the limb based on the comparison of the monitored venous flow rate with the venous flow rate threshold.

7. The blood filtration system of claim 1, wherein the withdrawal line is configured for communication with a first portion of the vasculature and the physiological sensors are configured to determine the venous flow rate of a second portion of the vasculature.

8. The blood filtration system of claim 1, further comprising the one or more physiological sensors.

9. The blood filtration system of claim 1, wherein:
the controller is configured to operate the cuff and increase the force applied to the limb based on a decrease in the monitored venous flow rate; and
the controller is configured to operate the cuff and decrease the force applied to the limb based on an increase in the monitored venous flow rate.

10-12. (canceled)

13. The blood filtration system of claim 1, wherein:
the one or more physiological sensors are configured to determine a venous pressure of the patient;
the controller is in communication with the one or more physiological sensors; and
the controller is configured to operate the cuff to adjust the force applied to the limb in correspondence with the determined venous pressure.

14. The blood filtration system of claim 13, wherein the controller is configured to adjust a cuff pressure corresponding to the force applied to the limb such that the cuff pressure is greater than or equal to the venous pressure.

15. The blood filtration system of claim 1, wherein the controller is configured to operate the cuff to allow arterial inflow into the limb in correspondence with inhibiting venous return from the limb.

16. The blood filtration system of claim 15, wherein the one or more physiological sensors are configured to determine one or more of a venous pressure or an arterial pressure of the patient, wherein the controller is configured to adjust the force applied to the limb to correspondingly apply a pressure that is greater than or equal to the venous pressure.

17. The blood filtration system of claim 16, wherein the controller is configured to adjust the force applied to the limb to correspondingly apply a pressure that is less than or equal to one or more of systolic arterial pressure or diastolic arterial pressure.

18. The blood filtration system of claim 1, wherein the limb of the patient includes an arm of the patient.

19. The blood filtration system of claim 1, wherein the limb of the patient includes a leg of the patient.

20. A blood filtration system, comprising:
one or more sensors configured to determine physiological parameters of a patient;
a cuff pump configured for communication with an adjustable cuff, wherein the adjustable cuff is configured to receive a fluid to inflate or deflate the adjustable cuff, and the cuff pump is configured to supply the fluid to the adjustable cuff;
a controller including:
a cuff module configured to operate the cuff pump;
a sensor interface configured to communicate with the one or more sensors;
a comparator configured to compare the physiological parameters of the patient to a physiological threshold; and
wherein the cuff module is configured to operate the cuff pump based on the comparison.

21. The blood filtration system of claim 20, further comprising the cuff.

22. The blood filtration system of claim 20, wherein the one or more sensors include a pulse oximeter configured to determine oxygen saturation of a patient.

23. The blood filtration system of claim 22, wherein the pump module is configured to operate the cuff pump based on a comparison of the oxygen saturation of the patient to an oxygen saturation threshold.

24. The blood filtration system of claim 22, wherein the pulse oximeter is configured for attachment to a patient distally with respect to the adjustable cuff.

25. The blood filtration system of claim 20, wherein the one or more physiological sensors include an ultrasonic transducer configured to determine flip of blood in vasculature of the patient.

26. The blood filtration system of claim 25, wherein the pump module is configured to operate the cuff pump based on a comparison of the flow rate of blood to a flow rate threshold.

27. The blood filtration system of claim 20, wherein the one or more physiological sensors are configured to determine one or more of force or pressure supplied to the adjustable cuff by the cuff pump.

28. The blood filtration system of claim 20, wherein:
the physiological parameters include one or more of venous pressure or arterial pressure of a patient;
the comparator is configured to compare the one or more venous pressure or arterial pressure to a pressure threshold; and
the cuff pump is configured to operate the cuff based on the comparison of the one or more venous pressure or arterial pressure to the pressure threshold.

29. The blood filtration system of claim 28, wherein the cuff pump is configured to adjust the force applied to the limb to correspondingly apply a pressure that is greater than or equal to the venous pressure.

30. The blood filtration system of claim 29, wherein the cuff pump is configured to adjust the force applied to the limb to correspondingly apply a pressure that is less than or equal to the arterial pressure.

31. A blood filtration system, comprising:
a withdrawal line and an infusion line configured to transmit blood from vasculature, wherein the withdrawal line and the infusion line are configured to couple with a filter, and the filter is configured to remove one or more plasma constituents from the blood of a patient;
an adjustable cuff configured to receive a portion of a limb of a patient, wherein:
the cuff is configured to engage with the limb to apply a force to the limb, and the engagement of the cuff with the limb is configured to inhibit flow of blood within the limb; and
wherein the withdrawal line is configured to communicate with vasculature located distally with respect to the cuff, and
the infusion line is configured to communicate with vasculature located proximally with respect to the cuff.
a controller configured to operate the cuff to adjust the force applied to the limb of the patient.

32. The blood filtration system of claim 31 wherein the cuff is configured to inhibit venous return within the limb.

33. The blood filtration system of claim 31, wherein:
the cuff is configured to inhibit venous return within the limb; and
the cuff is configured to permit arterial inflow into the limb.

34. The blood filtration system of claim 31, wherein:
the withdrawal line is configured to couple with a withdrawal catheter;
the infusion line is configured to couple with an infusion catheter; and
the withdrawal catheter is separate from the infusion catheter.
